SEQUENCE LISTING (15 sequences)

SEQ ID NO: 1 - protein, 140 aa, Artificial Sequence
  Description of Artificial Sequence note = synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 15 aa, Artificial Sequence
  Description of Artificial Sequence note = synthetic construct
GAPQEGILEDMPVDP

SEQ ID NO: 3 - protein, 15 aa, Artificial Sequence
  Description of Artificial Sequence note = synthetic construct
MPVDPDNEAYEMPSE

SEQ ID NO: 4 - protein, 15 aa, Artificial Sequence
  Description of Artificial Sequence note = synthetic construct
VTGVTAVAQKTVEGA

SEQ ID NO: 5 - protein, 8 aa, Artificial Sequence
  Description of Artificial Sequence note = synthetic construct
DYKDDDDK

SEQ ID NO: 6 - DNA, 1102 bp, Artificial Sequence
  Description of Artificial Sequence note = synthetic construct
gcgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaatcatatgaaaaagacagctatcgcgattgcagtggcactggctggtttcgctaccgttgcgcaagctgactacaaggacgacgatgacaagctttcctatgagctgacacagccaccctcagcgtctgggacccccgggcagagggtcaccatctcttgttctggaagcagctccaacatcggaagtaattatgtatactggtaccagcagctcccaggaacggcccccagactcctcatctataggaataatcagcggccctcaggggtccctgaccgattctctggctccaagtctggcacctcagcctccctggccatcagtgggctccggtccgaggatgaggctgattattactgtgcagcatgggatgacagtctgaatggtccggtattcggcggagggaccaagctcaccgtcctaggtgagggtaaatcttccggatctggttccgaatccaaagctagcgaggtgcagctggtgcagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtacctccccagagacactctactggggccagggcaccctggtcaccgtctcctcagtcgacccattcgtttctgaatatcaaggccaatcgtctgacctgcctcaacctcctgtcaatgctggcggcggctctgagggaggcggttccggtggtggctctgtcc

SEQ ID NO: 7 - DNA, 1101 bp, Artificial Sequence
  Description of Artificial Sequence note = synthetic construct
gatgcagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaatcatatgaaaaagacagctatcgcgattgcagtggcactggctggtttcgctaccgttgcgcaagctgactacaaggacgacgatgacaagctttcttctgagctgactcaggaccctgctgtgtctgtggccttgggacagacagtcaggatcacatgccaaggagacagcctcagaagctattatgcaagctggtaccagcagaagccaggacaggcccctatacttgtcatctatggtaacaataatcggccctcagggatcccagaccgattctctggctccagtctaggaaacactgcttccttgaccatcactggggctcaggcggacgatgaggctgactattactgtaactcccgggacagtagggctagacatgttttattcggcggagggaccaaggtgaccgtcctaggtgagggtaaatcttccggatctggttccgaatccaaagctagcgaggtgtagctggtgcagtctgggggaggcgtggtcaagcctggagggtccctgagactctcctgtgaagcctctggattcatactcagtgactattacatgacctggatccgccaggcaccagggaaggggctggagtggcttgcagtcattgatattactagtagttacacaaactacgcagactctgtgaagggccgcttcaccatctccagagacaacgccaagaactcagtgtatctgcaaatgaacagcctgagagccgaggacacggccgtgtattactgtgcgaggctggaatctggcttctttgactactggggccagggcaccctggtcaccgtctcctcagtcgacccattcgtttctgaatatcaaggccaatcgtctgacctgcctcaacctcctgtcaatgctggcggcggctctgagggaggcggttccggtggtggctctgtccgggga

SEQ ID NO: 8 - DNA, 1110 bp, Artificial Sequence
  Description of Artificial Sequence note = synthetic construct
cgatgcagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaatcatatgaaaaagacagctatcgcgattgcagtggcactggctggtttcgctaccgttgcgcaagctgactacaaggacgacgatgacaagcttcagtctgccctgactcagcctgcctccgtgtctgggtctcctggacagtcgatcaccatctcctgcactggaacctacagtgacattgctagttataaccacgtctcctggtaccaacaacacccaggcaaagcccccaaactcataatttttgaagtcgcttatcggccctcaggggtctcttatcgcttctctggctccaagtctggcaacacggcctccctgaccatctctgggctccaggctgaggacgaggctgattattactgcagttcatatacaaccagccgcacttatgtcttcggagctgggaccaagctcaccgtcctaggtgagggtaaatcttccggatctggttccgaatccaaagctagcgaggtgcagctggtgcagtctggaggaggcttgatccagcctggggggtccctgaaactctcctgtgcagcctctgggttcaccttcagtggctctgctatgcactgggtccgccaggcttccgggaaagggctggagtgggttggccgtattagaagcaaagctaacagttacgcgacagcatatgctgcgtcggtgaaaggcaggttcaccatctccagagatgattcaaagaacacggcgtatctgcaaatgaacagcctgaaaaccgaggacacggccgtgtattactgtactagacatcgaggagggcccattgactggggccagggcaccctggtcaccgtctcctcagtcgacccattcgtttctgaatatcaaggccaatcgtctgacctgcctcaacctcctgtcaatgctggcggcggctctgagggaggcggttccggtggtggctctgtccggga

SEQ ID NO: 9 - DNA, 1117 bp, Artificial Sequence
  Description of Artificial Sequence note = synthetic construct
cgatgcagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaatcatatgaaaaagacagctatcgcgattgcagtggcactggctggtttcgctaccgttgcgcaagctgactacaaggacgacgatgacaagcttcagtctgtgctgactcagccgccctcagtgtctggggccccaggtcagagggtcgccatctcctgcactgggaccagctccgacatcgggacaggttatgatgtaaattggtaccagcatcttccaggaacagcccccagactcctcatctatggtaatacctatcgaccctcaggggtccctgaccgattctctgcctccactggctccaagtctggcacctcagcctccctggtcatcactgacctccaggctgaagatgagggtgattattactgccagtcttttgacaacagcctgagaggttcggtgttcggcggagggaccaaggtgaccgtcctaggtgagggtaaatcttccggatctggttccgaatccaaagctagcgaggtgcagctggtgcagtctgggggaggcgtggtcaagcctggagggtccctgagactctcctgtgaagcctctggattcatactcagtgactattacatgacctggatccgccaggcaccagggaaggggctggagtggcttgcagtcattgatattactagtagttacacaaactacgcagactctgtgaagggccgcttcaccatctccagagacaacgccaagaactcagtgtatctgcaaatgaacagcctgagagccgaggacacggccgtgtattactgtgcgaggctggaatctggcttctttgactactggggccagggcaccctggtcaccgtctcctcagtcgacccattcgtttctgaatatcaaggccaatcgtctgacctgcctcaacctcctgtcaatgctggcggcggctctgagggaggcggttccggtggtggctctggttcc

SEQ ID NO: 10 - DNA, 1126 bp, Artificial Sequence
  Description of Artificial Sequence note = synthetic construct
gatgcagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaatcatatgaaaaagacagctatcgcgattgcagtggcactggctggtttcgctaccgttgcgcaagctgactacaaggacgacgatgacaagcttcagtctgccctgactcagcctcgctcagtgtccgggactcctggacagtcagtcaccatctcctgcactggaaccaacagtgatgttggtcgttatgactatgtctcgtggtaccaacagtattcagacaaagcccccaaactcatcatttatgatgactttaagcggccctcaggggtccctgatcgcttctctggctccaagtctggcaacacggcctccctgaccatctctgggctccaggctgaggatgaggctgattattactgcagctcatatacaagcagcagccgggtgttcggcggagggaccaaggtgaccgtcctaggtgagggtaaatcttccggatctggttccgaatccaaagctagcgaggtgcagctggtgcagtctgaagctgaggtgaggaagcctggggcctcagtgaaggtctcctgcaaggcttctggttttcactttaccagctacggtttcatgtgggtgcggcaggcccctggacaagggcttgagtggatgggttggatcagcgcttacaatggtaacacaaagtcggctcagaagttccagggcagactcaccatgaccactgacacatccacgaacacagcccacatggagttgatgagcctgagatctgacgacacggccgtgtattactgtgcgagagattctgcgactctaatagcctctcgacaggggggctctgactactggggccagggcaccctggtcaccgtctcctcagtcgacccattcgtttctgaatatcaaggccaatcgtctgacctgcctcaacctcctgtcaatgctggcggcggctctgagggaggcggttccggtggtggctctggtccgggga

SEQ ID NO: 11 - DNA, 1091 bp, Artificial Sequence
  Description of Artificial Sequence note = synthetic construct
cgatgcagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaatcatatgaaaaagacagctatcgcgattgcagtggcactggctggtttcgctaccgttgcgcaagctgactacaaggacgacgatgacaagcttgatgttgtgatgactcagtctccaggcaccctgtctttgtctccaggggaaagagccaccctctcctgcagggccagtcagagtgttagcatcgtctatttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcggtttattactgtcagcagtataatgactggttcactttcggcggagggaccaaagtggatatcaaagagggtaaatcttccggatctggttccgaatccaaagctagcgaggtgcagctggtgcagtctgggggaggcgtggtcaagcctggagggtccctgagactctcctgtgaagcctctggattcatactcagtgactattacatgacctggatccgccaggcaccagggaaggggctggagtggcttgcagtcattgatattactagtagttacacaaactacgcagactctgtgaagggccgcttcaccatctccagagacaacgccaagaactcagtgtatctgcaaatgaacagcctgagagccgaggacacggccgtgtattactgtgcgaggctggaatctggcttctttgactactggggccagggaaccctggtcaccgtctcctcagtcgacccattcgtttctgaatatcaaggccaatcgtctgacctgcctcaacctcctgtcaatgctggcggcggctctgagggaggcggttccggtggtggctctgtcc

SEQ ID NO: 12 - DNA, 1132 bp, Artificial Sequence
  Description of Artificial Sequence note = synthetic construct
gatgcagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaatcatatgaaaaagacagctatcgcgattgcagtggcactggctggtttcgctaccgttgcgcaagctgactacaaggacgacgatgacaagcttcagtctgtgctgactcagccgccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgagtggtgtggtattcggcggagggaccaagctgaccgtcctaggtgagggtaaatcttccggatctggttccgagtccaaagctagccaggtgcagctgcagtctggagctgaggtgaagaagcctggggcctcagtgaaagtctcctgcaaggcttctggttacacgtttaacactcatggtttcagctgggtacgacaggcccctggacaagggcttgagtggatgggatggatcagcgcatccaatggtaacacaaagtatccccagaacctccagggcagagtcaccatgactattgacacattcacgaccacagcatacctggaactgaggagcctgcgatctgacgacacggccgtgtattattgtgtgagagatagaacagattactatgttccggggacttttgatcccttatatgggccctttgattactggggccagggcaccctggtcaccgtctcctcagtcgacccattcgtttctgaatatcaaggccaatcgtctgacctgcctcaacctcctgtcaatgctggcggcggctctgagggaggcggttccggtggtggctctg

SEQ ID NO: 13 - DNA, 1112 bp, Artificial Sequence
  Description of Artificial Sequence note = synthetic construct
cgatgcagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaatcatatgaaaaagacagctatcgcgattgcagtggcactggctggtttcgctaccgttgcgcaagctgactacaaggacgacgatgacaagcttcagtctgccctgactcagcctgcctccgtgtctgggtctcctggacagtcgatcaccatctcctgcactggaaccagcagtgacgttggtacttataattatgtctcttggtatcaacaatatccaggcaaagcccccaaactcctgatttatgatgtcgttaagcggccctcaggggtttctaatcgcttctctggctccaagtctggcagcacggcctccctgaccatctctgggctccaggctgaggacgaggctgattattactgcagctcatatacaaccaggaacactcgggtattcggcggagggaccaagctcaccgtcctaggtgagggtaaatcttccggatctggttccgaatccaaagctagccaggttcagctggtgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaagaacttgggtggtaactccggtggggtggcctactggggccagggcaccctggtcaccgtctcctcagtcgacccattcgtttctgaatatcaaggccaatcgtctgacctgcctcaacctcctgtcaatgctggcggcggctctgagggaggcggttccggtggtggctctgtcc

SEQ ID NO: 14 - DNA, 1138 bp, Artificial Sequence
  Description of Artificial Sequence note = synthetic construct
cgatgcagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaatcatatgaaaaagacagctatcgcgattgcagtggcactggctggtttcgctaccgttgcgcaagctgactacaaggacgacgatgacaagcttcaggctgtgctcactcagccgtcttccctctctgcatctcctggagcatcagccagtctcacctgcaccttgcgcagtggcatcaatgttggtacctacaggatatactggtaccagcagaagccagggagtcctccccagtatctcctgaggtacaaatcagactcagataagcagcagggctctggagtccccagccgcttctctggatccaaagatgcttcggccaatgcagggattttactcatctctgggctccagtctgaggatgaggctgactattactgtatgatttggcacagcagcgcttatgtcttcggaactgggaccaagctgaccgtcctaggtgagggtaaatcttccggatctggttccgaatccaaagctagcgaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgagcggtagcagctcgtccttgggctactactactacggtatggacgtctggggccaagggaccacggtcaccgtctcctcagtcgacccattcgtttctgaatatcaaggccaatcgtctgacctgcctcaacctcctgtcaatgctggcggcggctctgagggaggcggttccggtggtggctctggt

SEQ ID NO: 15 - DNA, 1112 bp, Artificial Sequence
  Description of Artificial Sequence note = synthetic construct
cgatgcagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaatcatatgaaaaagacagctatcgcgattgcagtggcactggctggtttcgctaccgttgcgcaagctgactacaaggacgacgatgacaagcttcagtctgccctgactcagcctgcctccgtgtctgggtctcctggacagtcgatcaccatctcctgcactgaaaccggcagtgacgttggtggttatcactatgtctcctggtatcaacaccacccagacaaagcccccaaactcataatttatgatgactttaagcggccctcaggggtttctgatcgcttctctggctccaagtctggcaacacggcctccctgaccatctctgggctccgggctgaggacgaggctgattattactgcggctcatttacaagcagtgccccttggttgttcggcggagggaccaagctgaccgtcctaggtgagggtaaatcttccggatctggttccgaatccaaagctagcgaggtgcagctggtgcagtctgggggaggcttggtacagcctggggggtccctgagactctcctgtgcagcctctggattcacctttagcagttatgccatgagctgggtccgccaggctccagggaaggggctggagtgggtctcagcaattagtggtagtggtagtatcgcatactacgcagactccgtgaagggccggttcaccatctccagagacaattccaagaacacgctgtatctgcaaatgaacagcctgagagtcgaggacacggccgtatattactgtgcacgacgagctactggaacctcgtactactttgactactggggccagggcaccctggtcaccgtctcctcagtcgacccattcgtttctgaatatcaaggccaatcgtctgacctgcctcaacctcctgtcaatgctggcggcggctctgagggaggcggttccggtggtggctctgtcc

(19) United States
(12) Patent Application Publication
(10) Pub. No.: US 2008/0300204 A1
(43) Pub. Date: Dec. 4, 2008

(54) Alpha-Synuclein Antibodies and Methods Related Thereto

(75) Inventors: Howard J. Federoff, Bethesda, MD (US); Kathleen Maguire-Zeiss, Washington, DC (US); Mark Sullivan, Fairport, NY (US)

Correspondence Address: CLARK G. SULLIVAN;ARNALL GOLDEN GREGORY LLP, 171 17TH STREET NW, SUITE 2100, ATLANTA, GA, 30363, US

(73) Assignee: University of Rochester

(21) Appl. No.: 11/996,262

(22) PCT Filed: Jul. 19, 2006
(86) PCT No.: PCT/US06/27772

§ 371 Date: Aug. 14, 2008

Related U.S. Application Data
(60) Provisional application No. 60/700,565, filed on Jul. 19, 2005.

(51) Int. Cl. A61K 31/70; C07K 16/18; G01N 33/53; A61P 25/00; G01N 33/566

(52) U.S. Cl. 514/44; 530/387.1; 530/387.3; 530/388.1; 435/7.21; 436/501

(57) ABSTRACT

Disclosed are antibodies specific for alpha-synuclein conformers and methods related thereto. For example, disclosed are methods of diagnosing a neurodegenerative monitoring a neurodegenerative disease treatment using the disclosed antibodies. Assays, kits, and solid supports related to alpha-synuclein and antibodies specific for alpha-synuclein are also disclosed.

### CROSS-REFERENCE TO RELATED APPLICATIONS

This application claims benefit of U.S. Provisional Application No. 60/700,565, filed Jul. 19, 2005, which is hereby incorporated herein by reference in its entirety.

### STATEMENT REGARDING FEDERALLY SPONSORED RESEARCH

This invention was made with government support under Grant ______ awarded by the Dept. of Defense. The government has certain rights in the invention.

### BACKGROUND OF THE INVENTION

Parkinson's disease affects more than half a million Americans each year. Parkinson's disease is characterized by slowness of movement (bradykinesia), tremor at rest, rigidity of the extremities and neck, stooped posture, minimal facial expressions, problems swallowing (dysphagia), and a paucity of associated movements (e.g., arm swinging). Some patients also experience dementia associated with such abnormalities of motor function. Parkinson's disease is age-dependant and usually has a gradual onset between the ages of 50 and 70, progressing slowly until death 10 to 20 years later.

Oftentimes, the symptoms associated with Parkinson's disease can be similar to the symptoms of other neurodegenerative diseases. Also, the etiology of many neurodegenerative diseases, such as Parkinson's disease, is not fully understood. And currently, there are no known markers for identification of sporadic Parkinson's disease. Such difficulties can cause confusion and complications with diagnosing and treating patients with such neurodegenerative diseases.

Needed in the art are compositions and methods for differentiating, diagnosing, monitoring and treating a neurodegenerative disease such as Parkinson's disease. The subject matter disclosed herein addresses these and other needs.

### BRIEF SUMMARY OF THE INVENTION

In accordance with the purposes of the disclosed materials, compounds, compositions, articles, and methods, as embodied and broadly described herein, the disclosed subject matter, in one aspect, relates to compounds and compositions and methods for preparing and using such compounds and compositions. In another aspect, the disclosed subject matter relates to antibodies for alpha-synuclein. In still another aspect, the disclosed subject matter relates to methods of identifying and using such antibodies. In yet another aspect, the disclosed subject matter relates to methods of diagnosing a neurodegenerative disease (e.g., Parkinson's disease) in a subject, methods of monitoring a neurodegenerative disease progression in a subject. In another aspect, the of monitoring a neurodegenerative disease progression in a subject. In another aspect, the disclosed subject matter relates to methods of monitoring a response to a neurodegenerative disease treatment in a subject, methods of identifying a risk for a neurodegenerative disease in a test subject, and methods of differentially diagnosing a neurodegenerative disease in a test subject. In a further aspect, disclosed herein are diagnostic assays for a neurodegenerative disease. Also disclosed are methods of treating a neurodegenerative disease.

The advantages described below will be realized and attained by means of the elements and combinations particularly pointed out in the appended claims. It is to be understood that both the foregoing general description and the following detailed description are exemplary and explanatory only and are not restrictive.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the appended claims.

### BRIEF DESCRIPTION OF THE DRAWINGS

The accompanying Figures, which are incorporated in and constitute a part of this specification, illustrate several embodiments and, together with the description, illustrate the disclosed compositions and methods.

FIG. 1 is a schematic showing the construction, panning, and identification of scFvs recognizing different forms of α-Synuclein (SYN). A phage display library comprising human heavy and light chains was constructed to express on the surface of phage M13. The library was panned against monomeric, aggregated, and dopamine modified alpha-synuclein. Phage binding the proteins were enriched through successive cycles of binding, the scFvs moved into E. coli and the antibodies expressed.

FIG. 2 is a histogram showing the effect of a dopamine-quinone (DAQ)-specific single chain antibody, scFvDAQ6, on SYN-mediated cell death. MS9Dαsyn cells were grown in the absence (−Dox) and presence (+Dox) of doxycycline. Doxycycline induces SYN expression. Cells were transduced with HSV amplicons expressing either scFvs or beta-galactosidase as follows: HSVscFvDAQ6 or HSVscFvphe and HSVlac as controls (MOI=0.25). Twenty four hours later, the % of dead cells was calculated following propidium iodide treatment and cell sorting.

FIG. 3 shows analysis of SYN. FIG. 3A shows Coomassie blue stain. Human α-synuclein (SYN) was bacterially expressed and purified. A portion of SYN was further treated by incubating 1 mg/ml SYN at 33° C. with agitation (1,000 rpm) for 4 days in the absence or presence of 3.5 mM DA (dopamine). Five μg of either unmodified or modified SYN was subjected to SDS-PAGE (10%) and subsequently stained with Coomassie blue. *=stacking and resolving gel interface; arrow indicates monomeric SYN. FIG. 3B shows SYN western blot analysis. Two μg of either untreated or treated SYN were subjected to SDS-PAGE (10%), transferred to PVDF membrane and immunoblot analyzed utilizing anti-SYN antibodies (BD Bioscience mouse anti-SYN antibodies; 1:1000) or no primary antibody (No 1°). Immunocomplexes were visualized following incubation with anti-mouse HRP (1:2500) and enhanced chemiluminescence. *=stacking and resolving gel interface; arrow indicates monomeric SYN. FIG. 3C shows Atomic force microscopy. SYN samples were subjected to atomic force microscopy (AFM) and images captured using a Digital Instruments NanoScope as described in Materials and Methods. Z height scale is given below the untreated sample (white=20 nm and black=0 nm); Untreated=native/monomeric SYN; DA/1,000 rpm=SYN treated with 3.5 mM DA at 33° C. and agitation; 1,000 rpm=SYN incubated at 33° C. and agitation.

FIG. 4 shows panning, identification, expression and purification of scFvs. A combinatorial phage display library expressing human immunoglobulin heavy and light chain variable regions was panned for reactivity to various antigens. Following reamplification, scFvs were PCR subcloned into pCOMB3x appending a hexahistidine sequence to the 3′ end and a FLAG™ sequence to the 5′ end. ScFvs were expressed in E. coli following either IPTG induction or by growth of cultures in a low phosphate containing medium to induce expression from the phoA promoter. Expressed scFvs were purified using metal affinity chromatography (TALON™). ScFv purity was evaluated following protein staining and western blot analysis (right). Five and 0.5 μg of scFv was subjected to SDS-PAGE (10%). The 5 μg sample was subsequently stained with Coomassie blue, and the 0.5 μg sample was transferred to PVDF membrane, subjected to western blot analysis using mouse anti-M2 (FLAG™) antibodies (1:2500), followed by goat antimouse HRP (1:200) and visualization using enhanced chemiluminescence. The binding ability of purified scFv was tested in an ELISA. Microtiter wells were coated with DA-modified SYN (0.5 μg/well; SYN/DA/1,000 rpm for 4 days at 33° C.) and reacted with scFv6 (100-0.0013 μg/ml). Antigen:antibody complexes were visualized after incubation with a secondary antibody conjugated to horseradish peroxidase (1:1000; anti-HA-HRP), exposure to TMB peroxidase substrate and measurement of the chromogenic signal at 450 nm. Panning schematic was adapted from Barbas (Barbas, C. F., 2001).

FIG. 5 shows ScFv15 specificity. FIG. 5A shows ScFv15 ELISA. Microtiter wells were coated with purified and modified proteins as indicated (DA-modified SYN; 0.5 μg/well; protein/(+/−)DA/1,000 rpm for 4 days at 33° C.) and reacted with scFv15 (0.03 μg/well). Antigen:antibody complexes were visualized after incubation with a secondary antibody conjugated to horseradish peroxidase (1:1000; anti-FLAG-HRP), exposure to TMB peroxidase substrate and measurement of the chromogenic signal at 450 nm. FIG. 5B shows ScFv15 western blot analysis. ScFv15 was used as a primary antibody to probe for various conformers of SYN. Two μg of protein was subjected to SDSPAGE, transferred to PVDF membrane and probed with ScFv15 (1:30; 0.35 mg/ml). Antigen:antibody complexes were visualized following incubation with anti-FLAG-HRP (1:1000) and enhanced chemiluminescence. Arrow indicates monomeric SYN.

FIG. 6 shows ScFv3 specificity. FIG. 6A shows ScFv3 ELISA. Microtiter wells were coated with purified and modified proteins as indicated (DA-modified SYN; 0.5 μg/well; protein/(+/−) DA/1,000 rpm for 7 days at 33° C.) and reacted with scFv3 (0.03 μg/well). Antigen:antibody complexes were visualized after incubation with a secondary antibody conjugated to horseradish peroxidase (1:1000; anti-HA-HRP), exposure to TMB peroxidase substrate and measurement of the chromogenic signal at 450 nm. FIG. 6B shows ScFv3 western blot analysis. ScFv3 was used as a primary antibody to probe for various conformers of SYN (+/−DA/1,000 rpm for 7 days at 33° C.). Five μg of protein was subjected to SDS-PAGE, transferred to PVDF membrane and probed with scFv3 (1:1000; 26 mg/ml). Antigen:antibody complexes were visualized following incubation with anti-HA-HRP (1:1000) and enhanced chemiluminescence. Arrow indicates monomeric SYN; * indicates interface between stacking and resolving gel.

FIG. 7 shows ScFv6 specificity. FIG. 7A shows ScFv6 ELISA. Microtiter wells were coated with purified and modified proteins as indicated (DA-modified SYN; 0.5 μg/well; protein/(+/−)DA/1,000 rpm for 7 days at 33° C.) and reacted with scFv6 (0.03 μg/well). Antigen:antibody complexes were visualized after incubation with a secondary antibody conjugated to horseradish peroxidase (1:1000; anti-HA-HRP), exposure to TMB peroxidase substrate and measurement of the chromogenic signal at 450 nm. FIG. 7B shows ScFv6 western blot analysis. ScFv6 was used as a primary antibody to probe for various conformers of SYN (+/−DA/1,000 rpm for 7 days at 33° C.). Five μg of protein was subjected to SDS-PAGE, transferred to PVDF membrane and probed with scFv6 (1:100; 2.0 mg/ml). Antigen:antibody complexes were visualized following incubation with anti-HA-HRP (1:1000) and enhanced chemiluminescence. Arrow indicates monomeric SYN; * indicates interface between stacking and resolving gel.

FIG. 8 shows linear peptide mapping of scFvs. Streptavidin coated microtiter wells were incubated with biotin-conjugated synthetic 15 amino acid overlapping SYN peptides spanning the entire sequence (50 μg/well) and subsequently reacted with scFv15, scFv14, scFv3 or scFv6 (0.03 μg/well). Antigen:antibody complexes were visualized after incubation with a secondary antibody conjugated to horseradish peroxidase (1:1000; anti-HA-HRP), exposure to TMB peroxidase substrate and measurement of the chromogenic signal at 450 nm.

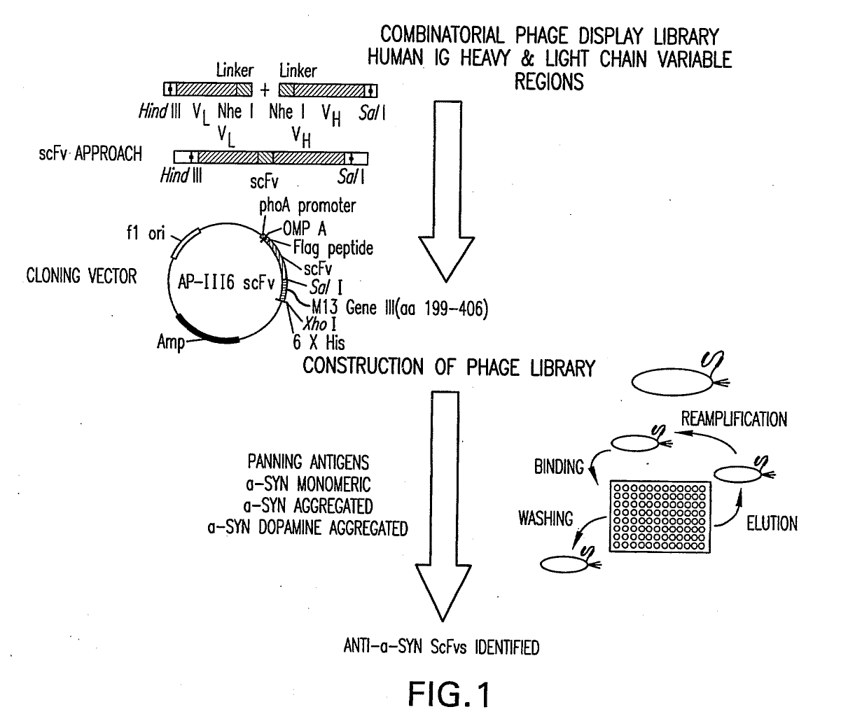

### DETAILED DESCRIPTION OF THE INVENTION

The materials, compounds, compositions, articles, devices, and methods described herein can be understood more readily by reference to the following detailed description of specific aspects of the disclosed subject matter and the Examples included herein and to the Figures.

Before the present compounds, compositions, articles, devices, and methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular aspects only and is not intended to be limiting.

Also, disclosed herein are the materials, compounds, and components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular protein (e.g., antibody) is disclosed and discussed and a number of modifications that can be made to a number of molecules (e.g., amino acids) are discussed, specifically contemplated is each and every combination and permutation of the protein and the modifications that are possible unless specifically indicated to the contrary. Thus, if a class of molecules A, B, and C are disclosed as well as a class of molecules D, E, and F, and an example of a combination molecule A-D is disclosed, then even if each is not individually recited, each is individually and collectively contemplated, meaning combinations A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Further, throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### DEFINITIONS

In this specification and in the claims which follow, reference will be made to a number of terms which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the specification and the claims, the singular forms “a,” “an,” and “the” include plural referents unless the context clearly dictates otherwise. Thus, for example, reference to “a sample” includes mixtures of two or more such samples, reference to “an antibody” includes mixtures of two or more antibodies, reference to “the subject” includes two or more subjects, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15.

“Optional” or “optionally” means that the subsequently described event or circumstance may or may not occur, and that the description includes instances where said event or circumstance occurs and instances where it does not.

As used herein, the terms “subject” and “patient” are used interchangeably and mean an individual. Thus, “subject” or “patient” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” or “patient” can also include a mammal, such as a primate. In one particular aspect, a “subject” or “patient” can be a human.

As used herein, “sample” refers to any biological material obtained from a subject or patient. In one aspect, a sample can comprise blood, cerebrospinal fluid (“CSF”), or urine. In other aspects, a sample can comprise whole blood, plasma, leukocytes enriched from blood samples, and cultured cells (e.g., leukocytes from a subject). A sample can also include a biopsy or tissue sample including neural tissue. In still other aspects, a sample can comprise whole cells and/or a lysate of the cells. Examples of cells include, but are not limited to, leukocytes such as neutrophils, monocytes, basophils, lymphocytes, eosinophils, or any combination thereof. In another particular aspect, a sample can comprise a leukocyte or substantially pure population of leukocytes or a lysate thereof. The term “substantially pure” with respect to a population of leukocytes or lysates thereof is intended to refer to a sample that contains less than about 1%, less than about 5%, less than about 7%, less than about 10%, less than about 12%, less than about 15%, less than about 20%, less than about 25%, or less than about 30% of cells other than leukocytes, based on the total number of cells in the sample. In a specific example, a sample can comprise lymphocytes, a substantially pure population of lymphocytes, or a lysate of a substantially pure population of lymphocytes. Optionally, the leukocytes can be enriched for a selected type. For example, the leukocyte population can be enriched for lymphocytes and used in the methods described herein. Enrichment can be accomplished using cell sorting techniques like FACS.

Blood samples can be collected by methods known in the art. In one aspect, the pellet can be resuspended by vortexing at 4° C. in 200 μL buffer (20 mM Tris, pH. 7.5, 0.5% Nonidet, 1 mM EDTA, 1 mM PMSF, 0.1M NaCl, 1× Sigma Protease Inhibitor, and 1× Sigma Phosphatase Inhibitors 1 and 2). The suspension can be kept on ice for 20 minutes with intermittent vortexing. After spinning at 15,000×g for 5 minutes at about 4° C., aliquots of supernatant can be stored at about −70° C.

There are a variety of compositions disclosed herein that are amino acid based, including for example alpha-synuclein and antibodies specific for alpha-synuclein. Thus, as used herein, “amino acid” means the typically encountered twenty amino acids which make up polypeptides. In addition, it further includes less typical constituents which are both naturally occurring, such as, but not limited to formylmethionine and selenocysteine, analogs of typically found amino acids, and mimetics of amino acids or amino acid functionalities. Non-limiting examples of these and other molecules are discussed herein.

As used herein, the terms “peptide” and “protein” refer to a class of compounds composed of amino acids chemically bound together. Non-limiting examples of these and other molecules are discussed herein. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids and small and large peptides, including polypeptides and proteins. The terms “peptide,” “polypeptide,” and “protein” are used interchangeably herein.

“Deletion,” as used herein, refers to a change in an amino acid sequence in which one or more amino acid residues are absent relative to the reference sequence.

“Insertion” or “addition,” as used herein, refers to a change in an amino acid sequence resulting in the addition of one or more amino acid residues as compared to the reference sequence.

“Substitution,” as used herein, refers to the replacement of one or more amino acids by one or more different amino acids in a reference sequence

Also, a variety of sequences are provided herein and these and others can be found in Genbank, at www.pubmed.gov. Those of skill in the art understand how to resolve sequence discrepancies and differences and to adjust the compositions and methods relating to a particular sequence to other related sequences.

Reference will now be made in detail to specific aspects of the disclosed materials, compounds, compositions, articles, devices, and methods, examples of which are illustrated in the accompanying Examples and Figures.

### Alpha-Synuclein

Parkinson's disease is an age-dependent neurodegenerative disease with no known etiology. It is believed that sporadic Parkinson's disease results from a combination of genetic vulnerability and environmental insults. It is further believed that Parkinson's disease while triggered by disparate mechanisms follows a shared pathophysiologic pathway. One shared node is the involvement of alpha-synuclein. Linkage of this protein with Parkinson's disease pathogenesis has been established by the identification of both point mutations and triplication of the gene in familial cases, the localization of alpha-synuclein to Lewy bodies, one of the hallmark pathological features of Parkinson's disease, and the correlation of alpha-synuclein expression and disease pathology in neurotoxic models of Parkinson's disease. Further evidence indicates that particular forms of alpha-synuclein (e.g., misfolded and alpha-synuclein bonded dopamine) are involved in sporadic disease.

Alpha-synuclein exists in its native form as a random coil; however, changes in pH, molecular crowding, heavy metal content, and dopamine levels all affect protein conformation. Changes in conformation to protofibrillar, fibrillar, and aggregate moieties are thought to regulate protein toxicity. Increasing evidence indicates that dopamine-adducted alpha-synuclein has a faster time course to fibril formation compared to non-adducted protein. Furthermore, dopamine in the background of alpha-synuclein overexpression is toxic. That is, while focusing on the unique properties of the dopamine neuron environment, i.e., dopamine manufacture, release, and uptake, evidence disclosed herein indicates that in the presence of alpha-synuclein, dopamine renders cells more vulnerable and likely to succumb. Currently there are no available tests for monitoring alpha-synuclein conformers in blood, CSF, or urine.

In this specification, the term “alpha synuclein” is used to specifically refer to the native monomer form of alpha-synuclein. The term “alpha-synuclein” is also used to generally identify other conformers of alpha-synuclein, for example, alpha-synuclein bonded to dopamine-quinone (DAQ) and oligomers or aggregates of alpha-synuclein. The term “alpha-synuclein” is also used to refer collectively to all types and forms of alpha-synuclein. The protein sequence for human alpha-synuclein is MDVFMKGLSKAKEGVVAAAEKTKQGVAEAAGKTKEGVLYVGSKTKEGVVHGV ATVAEKTKEQVTNVGGAVVTGVTAVAQKTVEGAGSIAAATGFVKKDQLGKNEEG APQEGILEDMPVDPDNEAYEMPSEEGYQDYEPEA (SEQ ID NO:1) (swissprot: locus SYUA_HUMAN, accession number P37840).

### Antibodies to Alpha-Synuclein

Disclosed herein are compositions and methods that can be used to identify antibodies to alpha-synuclein in samples. By “antibodies to alpha-synuclein” and “anti-alpha-synuclein” is meant specifically, generally, and collectively, antibodies to the native form of alpha-synuclein, dopamine-adducted alpha-synuclein, and oligomeric or aggregated alpha-synuclein. Provided herein are antibodies selective for native, dopamine-quinone bonded, and oligomeric forms.

The disclosed anti-alpha-synuclein antibodies can be used to screen for the presence of alpha-synuclein in samples, for example, by using ELISA-based or surface adapted assay. In one aspect, disclosed herein are conformation-specific single chain antibodies that can be used to screen human blood, CSF, and urine. The methods and compositions disclosed herein can aid in Parkinson's disease diagnosis and can be used to monitor disease progression and therapeutic efficacy.

In one aspect, disclosed herein are antibodies that specifically bind alpha-synuclein and epitopes thereof and to various conformations of alpha-synuclein and epitopes thereof. For example, disclosed herein are antibodies that specifically bind alpha-synuclein, alpha-synuclein in its native monomer form, alpha-synuclein bonded to dopamine quinone, and oligomeric or aggregated alpha-synuclein. As used herein, reference to an antibody that “specifically binds” or “selectively binds” alpha-synuclein refers to an antibody that does not bind other unrelated proteins. In one example, an anti-alpha-synuclein antibody disclosed herein can bind alpha-synuclein or an epitope thereof and show no binding above about 1.5 times background for other proteins. Reference to an antibody that “specifically binds” or “selectively binds” alpha-synuclein conformer refers to an antibody that does not bind all conformations of alpha-synuclein, i.e., does not bind at least on other alpha-synuclein conformer. For example, disclosed herein are antibodies that can distinguish among alpha-synuclein conformational forms (conformers) and, thus, bind selectively with one (or more) conformers but not another.

An antibody disclosed herein can be a polyclonal antibody or a monoclonal antibody, which can be generated by techniques that are well known in the art. As used herein, the term “epitope” is meant to include any determinant capable of specific interaction with the disclosed alpha-synuclein antibodies. Epitopic determinants usually consist of chemically active surface groupings of molecules such as amino acids or sugar side chains and usually have specific three dimensional structural characteristics, as well as specific charge characteristics.

Immunoglobulins

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies (see Kabat, et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

Antibody Fragments

The term “antibody” as used herein is also meant to include intact molecules as well as fragments thereof, such as, for example, Fab and F(ab′)₂, which are capable of binding the epitopic determinant.

As used herein, the term “antibody or fragments thereof” encompasses chimeric antibodies and hybrid antibodies, with dual or multiple antigen or epitope specificities, and fragments, such as F(ab′)₂, Fab′, Fab, Fv and the like, including hybrid fragments. Thus, fragments of the antibodies that retain the ability to bind their specific antigens are provided. For example, fragments of antibodies which maintain alpha-synuclein binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (see Harlow and Lane, Antibodies, A Laboratory Manual, Cold Spring Harbor Publications, New York, (1988)).

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

Alternatively, unprotected peptide segments are chemically linked where the bond formed between the peptide segments as a result of the chemical ligation is an unnatural (non-peptide) bond (Schnolzer, et al., Science, 256:221 (1992)). This technique has been used to synthesize analogs of protein domains as well as large amounts of relatively pure proteins with full biological activity (deLisle Milton, et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with alpha-synuclein. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, e.g., New England Biolabs Product Catalog, 1996, p. 164). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen (Zoller, et al., Nucl Acids Res 10:6487-500 (1982); Zoller, et al., Curr Op Biotech 3:348-52 (1992), which are incorporated by reference herein at least for their teachings of antibody preparation).

In one aspect, an antibody disclosed herein is a single chain antibody (scFv). Techniques can be adapted for the production of single-chain antibodies specific to an antigenic protein disclosed herein (e.g., alpha-synuclein) (see e.g., U.S. Pat. No. 4,946,778). In addition, methods can be adapted for the construction of F(ab) expression libraries (see, e.g., Huse, et al., Science 246:1275-81 (1989)) to allow rapid and effective identification of monoclonal F(ab) fragments with the desired specificity for a protein or derivatives, fragments, analogs or homologs thereof. Antibody fragments that contain the idiotypes to a protein antigen can be produced by techniques known in the art including, but not limited to: (i) an F((ab′))(2) fragment produced by pepsin digestion of an antibody molecule; (ii) an Fab fragment generated by reducing the disulfide bridges of an F((ab′))(2) fragment; (iii) an F(ab) fragment generated by the treatment of the antibody molecule with papain and a reducing agent; and (iv) F(v) fragments.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

Hybrid Antibodies

As used herein, the term “hybrid antibody” refers to an antibody wherein each chain is separately homologous with reference to a mammalian antibody chain, but the combination represents a novel assembly so that two different antigens are recognized by the antibody. In hybrid antibodies, one heavy and light chain pair is homologous to that found in an antibody raised against one antigen recognition feature, e.g., epitope, while the other heavy and light chain pair is homologous to a pair found in an antibody raised against another epitope. This results in the property of multi-functional valency, i.e., ability to bind at least two different epitopes simultaneously. Such hybrids can be formed by fusion of hybridomas producing the respective component antibodies, or by recombinant techniques. Such hybrids may, of course, also be formed using chimeric chains.

Antibody Conjugates

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference. An antibody (or fragment thereof) can be conjugated to a therapeutic moiety such as a cytotoxin, a therapeutic agent, or a radioactive metal ion.

In one aspect, the conjugates disclosed herein can be used for modifying a given biological response. For example, a therapeutic moiety can be a protein or polypeptide possessing a desired biological activity. Such proteins can include, for example, a toxin such as abrin, ricin A, pseudomonas exotoxin, or diphtheria toxin; a protein such as tumor necrosis factor, [agr]-interferon, [bgr]-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

The antibody conjugates can be bound to a substrate or labeled with a detectable moiety or both bound and labeled. The detectable moieties contemplated with the disclosed compositions and methods include fluorescent, enzymatic, and radioactive markers.

Techniques for conjugating such moieties to antibodies are well known, see, e.g., Arnon, et al., in Monoclonal Antibodies And Cancer Therapy, Reisfeld, et al. (eds.), pp. 243-56 (Alan R. Liss, Inc. 1985); Hellstrom et al., in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies '84: Biological And Clinical Applications, Pinchera, et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985), and Thorpe, et al., “The Preparation And Cytotoxic Properties Of Antibody-Toxin Conjugates,” Immunol Rev 62:119-58 (1982). Alternatively, an antibody can be conjugated to a second antibody to form an antibody heteroconjugate as described by Segal in U.S. Pat. No. 4,676,980.

Monoclonal Antibodies

In one aspect, an antibody disclosed herein is a monoclonal antibody. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity. (See, U.S. Pat. No. 4,816,567 and Morrison, et al., Proc Natl Acad Sci USA, 81:6851-6855 (1984)).

Monoclonal antibodies can be prepared using hybridoma methods, such as those described by Kohler and Milstein, Nature, 256:495 (1975) or Harlow and Lane, Antibodies, A Laboratory Manual. Cold Spring Harbor Publications, New York, (1988), which are incorporated by reference herein at least for their teachings of monoclonal antibodies an their preparation. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. In one example, the immunizing agent comprises alpha-synuclein. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of alpha-synuclein expressed as a fusion protein with human IgG1 is injected into the host animal according to methods known in the art (e.g., Kilpatrick, et al., Hybridoma 17(6):569-76 (1998); Kilpatrick, et al., Hybridoma 19(4):297-302 (2000), which are incorporated by referenced herein at least for the methods of antibody production).

An alternate approach to immunizations with either purified protein or DNA is to use antigen expressed in baculovirus. The advantages of this system include ease of generation, high levels of expression, and post-translational modifications that are highly similar to those seen in mammalian systems. Use of this system involves expressing domains of alpha-synuclein as fusion proteins. The antigen is produced by inserting a gene fragment in-frame between the signal sequence and the mature protein domain of the alpha-synuclein antibody nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, New York, pp. 59-103 (1986)). Immortalized cell lines are usually transformed mammalian cells, including myeloma cells of rodent, bovine, equine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, amino pterin, and thymidine (“HAT medium”), which substances prevent the growth of HGPRT-deficient cells. Suitable immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, I. Immunol., 133:3001 (1984); Brodeur, et al., Monoclonal Antibody Production Techniques and Applications, Marcel Dekker, Inc., New York, pp. 51-63 (1987)). The culture medium in which the hybridoma cells are cultured can then be assayed for the presence of monoclonal antibodies directed against alpha-synuclein. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art and are described in Harlow and Lane, Antibodies, A Laboratory Manual, Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones can be isolated or purified from the culture medium or ascites fluid by conventional immunoglobulin purification procedures such as, for example, protein A-Sepharose, protein Q hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for alpha-synuclein and another antigen-combining site having specificity for a different antigen.

Verification of the epitope that the monoclonal antibody recognizes can be performed as follows. First, various partial structures of the molecule that the monoclonal antibody recognizes are prepared. The partial structures are prepared by the method wherein various partial peptides of the molecule are synthetically prepared by known oligopeptide synthesis technique, or the method wherein DNA encoding the desired partial polypeptide is incorporated in a suitable expression plasmid, and is expressed in a suitable host, such as E. coli, to produce the peptides. Generally, both methods are frequently used in combination for the above object. For example, a series of polypeptides having appropriately reduced lengths, working from the N-terminus of the antigen protein, can be prepared by established genetic engineering techniques. By establishing which fragments react with the antibody, an approximate idea of the epitope site is obtained.

The epitope is more closely identified by synthesizing a variety of smaller oligopeptides corresponding thereto or mutants of the peptide using established oligopeptide synthesis techniques to determine a binding property of the peptides to the anti-alpha-synuclein monoclonal antibody, for example, which is a basis for preparation of the antibody of the disclosed subject matter and a competitive inhibition of binding of the peptide to an antigen with the monoclonal antibody. Commercially available kits, such as the SPOTs Kit (Genosys Biotechnologies, Inc.) and a series of multipin peptide synthesis kits based on the multipin synthesis method (Chiron Corp.) can be conveniently used to obtain a large variety of oligopeptides.

Antibody molecules are purified by known techniques illustratively including amino absorption or amino affinity chromatography, chromatographic techniques such as high pressure liquid chromatography, or a combination thereof.

Human or Humanized

In one aspect, an antibody disclosed herein can be a humanized antibody. For example, the antibodies can be generated in other species and “humanized” for administration in humans. As used herein, the terms “human” and “humanized,” in relation to antibodies, relate to any antibody which is expected to elicit a therapeutically tolerable weak immunogenic response in a human subject. Humanized forms of non-human (e.g., murine) antibodies are chimeric immunoglobulins, immunoglobulin chains or fragments thereof (such as Fv, Fab, Fab′, F(ab′)₂, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones, et al., Nature 321:522-5 (1986); Riechmann, et al., Nature 332:323-7 (1988); and Presta, Curr Op Struct Biol 2:593-6 (1992), which are incorporated by reference herein at least for their teachings of humanized antibodies).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones, et al., Nature 321:522-5 (1986); Riechmann, et al., Nature 332:323-7 (1988); Verhoeyen, et al., Science 239:1534-6 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims, et al., J Immunol 151:2296 (1993) and Chothia, et al., J Mol Biol 196:901 (1987)). Another method uses a particular framework derived from the consensus sequence of all human antibodies of a particular subgroup of light or heavy chains. The same framework may be used for several different humanized antibodies (Carter, et al., Proc Natl Acad Sci USA 89:4285 (1992); Presta, et al., J Immunol 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis, of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see WO 94/04679).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits, et al., Proc Natl Acad Sci USA 90:2551-255 (1993); Jakobovits, et al., Nature 362:255-258 (1993); Bruggemann, et al., Year in Immuno 7:33 (1993)). Human antibodies can also be produced in phage display libraries (Hoogenboom, et al., J Mol Biol 227:381 (1991); Marks, et al., J Mol Biol 222:581 (1991)). The techniques of Cote, et al., and Boerner, et al., are also available for the preparation of human monoclonal antibodies (Cole, et al., Monoclonal Antibodies and Cancer Therapy, Alan R. Liss, p. 77 (1985); Boerner, et al., J Immunol 147(1):86-95 (1991), which are incorporated by reference herein at least for their teachings of human antibodies).

Antibody Modifications

The disclosed methods and compositions provide for an alpha-synuclein antibody, a humanized or fully human anti-alpha-synuclein antibody, heavy and light chain immunoglobulins and humanized or fully human heavy and light chain immunoglobulins. Also, disclosed herein are nucleic acids that encode the antibodies and heavy and light chains, vectors comprising those nucleic acids, and cells comprising the vectors. Certain truncations of these proteins or genes perform the regulatory or enzymatic functions of the full sequence protein or gene. For example, the nucleic acid sequences coding therefor can be altered by substitutions, additions, deletions or multimeric expression that provide for functionally equivalent proteins or genes. Due to the degeneracy of nucleic acid coding sequences, other sequences which encode substantially the same amino acid sequences as those of the naturally occurring proteins can be used in the practice of the disclosed subject matter. These include, but are not limited to, nucleic acid sequences including all or portions of the nucleic acid sequences encoding the above polypeptides, which are altered by the substitution of different codons that encode a functionally equivalent amino acid residue within the sequence, thus producing a silent change. It is appreciated that the nucleotide sequence of an immunoglobin according to the disclosed compositions and methods tolerates sequence homology variations of up to 25% as calculated by standard methods (“Current Methods in Sequence Comparison and Analysis,” Macromolecule Sequencing and Synthesis, Selected Methods and Applications, pp. 127-149 (1998), Alan R. Liss, Inc.) so long as such a variant forms an operative antibody which recognizes alpha-synuclein. For example, one or more amino acid residues within a polypeptide sequence can be substituted by another amino acid of a similar polarity which acts as a functional equivalent, resulting in a silent alteration. Substitutes for an amino acid within the sequence may be selected from other members of the class to which the amino acid belongs (i.e., a conservative substitution). For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Also included in the compositions and methods disclosed herein are proteins or fragments or derivatives thereof which are differentially modified during or after translation, e.g., by glycosylation, proteolytic cleavage, linkage to an antibody molecule or other cellular ligands, etc. In addition, the recombinant vector encoding nucleic acid sequences of the antibodies disclosed herein can be engineered so as to modify processing or expression of a vector. Other modifications can be made in either the nucleic acid or amino acid sequence without reducing or without substantially reducing apoptosis activity in the antibody. Such modifications can occur in the CDRs or non-CDR regions using techniques routine in the art. See, e.g., Yang, et al., J Mol Biol 254:392-403 (1995), which is hereby incorporated by reference in its entirety for methods of CDR walking mutagenesis.

The CDRs have the highest variability in amino acid sequence with the antibody. The portions of the variable region that are not part of a CDR are called “framework regions” (“FR” regions) and generally play a role in maintaining CDR structure. In one aspect, all the CDRs from a given antibody are grafted into an acceptor antibody, in order to preserve the binding region for the alpha-synuclein. It is appreciated that grafting a portion of the total amount of CDRs into a donor is operative herein. It is understood that grafting generally entails the replacement, residue for residue, of one amino acid or region, for another. However, occasionally, especially with the transfer of a region, one or more residues may be added or omitted or substituted therefor, as desired, and that such deletions and insertions, as well as appropriate replacements and inversions, are within the skill of those in the art.

ScFvs

Also disclosed are single-chain antibodies (scFvs) that can distinguish different SYN conformers. ScFvs are composed of the minimal antibody binding site formed by non-covalent association of the VH and VL variable domains joined by a flexible polypeptide linker (Haidaris, C. G., et al., 2001; Malone, J. and M. A. Sullivan, 1996). Furthermore, scFvs can be produced in large quantities and represent a renewable source of antibody. Different SYN conformers have been utilized to pan a human phage library for anti-SYN-conformer-specific scFvs.

Methods for the production of single-chain antibodies are well known to those of skill in the art. The skilled artisan is referred to U.S. Pat. No. 5,359,046 (incorporated herein by reference) for such methods. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation. See, e.g., Huston, et al., Methods in Enzym 203:46-121 (1991), which is incorporated herein by reference. These Fv's lack the constant regions (Fc) present in the heavy and light chains of the native antibody.

Methods of making scFv antibodies have been described in Huse et al., 1989. Science 246:1275-1281; Ward et al., 1989. Nature 341:544-546; Vaughan et al., 1996. Nature Biotech. 14:309-314, which are incorporated by reference herein in their entirety for the teaching of scFv production. In brief, mRNA from B-cells is isolated and cDNA is prepared. The cDNA is amplified by well known techniques, such as PCR, with primers specific for the variable regions of heavy and light chains of immunoglobulins. The PCR products are purified by, for example, agarose gel electrophoresis, and the nucleic acid sequences are joined. If a linker peptide is desired, nucleic acid sequences that encode the peptide are inserted between the heavy and light chain nucleic acid sequences. The sequences can be joined by techniques known in the art, such as blunt end ligation, insertion of restriction sites at the ends of the PCR products or by splicing by overlap extension (Chowdhury et al., 1997. Mol. Immunol 34:9). After amplification, the nucleic acid which encodes the scFv is inserted into a vector, again by techniques well known in the art. Preferably, the vector is capable of replicating in prokaryotes and of being expressed in both eukaryotes and prokaryotes.

Panning can be performed by any of several methods. A protocol for performing panning using cells is set forth in the Examples, below. Panning can also be performed on a solid surface by coating the solid surface with SYN confomers and incubating the phage on the surface for a suitable time under suitable conditions. Conveniently, the surface can be a magnetic bead. The unbound phage are washed off the solid surface and the bound phage are eluted.

Finding the antibody with the highest affinity is dictated by the efficiency of the selection process and depends on the number of clones that can be screened and the stringency with which it is done. Typically, higher stringency corresponds to more selective panning. If the conditions are too stringent, however, the phage will not bind. After one round of panning, the phage that bind to SYN conformer-coated plates or to cells expressing SYN confomers on their surface are expanded in E. coli and subjected to another round of panning. In this way, an enrichment of many fold occurs in 3 rounds of panning. Thus, even when enrichment in each round is low, multiple rounds of panning will lead to the isolation of rare phage and the genetic material contained within which encodes the scFv with the highest affinity or one which is better expressed on phage.

Regardless of the method of panning chosen, the physical link between genotype and phenotype provided by phage display makes it possible to test every member of a cDNA library for binding to antigen, even with large libraries of clones.

Examples SYN-conformer-specific scFvs include scFv3 (SEQ ID NO:6), scfv4 (SEQ ID NO:7), scFv5 (SEQ ID NO:8), scFv6 (SEQ ID NO:9), scfv7 (SEQ ID NO:10), scfv8 (SEQ ID NO:11), scFv10 (SEQ ID NO:12), scFv14 (SEQ ID NO:13), scFv15 (SEQ ID NO:14), scfv16 (SEQ ID NO:15).

### Solid Supports

Disclosed herein are solid supports (including, stable and mobile forms) wherein at least one address on one the solid support is an alpha-synuclein antibody as disclosed herein. Also disclosed are solid supports wherein at least one address is the sequence or portion of sequence set forth in any of the peptide sequences disclosed herein. Also disclosed are solid supports wherein at least one address is a variant of the antibodies or sequences or portions thereof as set forth herein. Solid supports include stable supports like slides, chips, microarrays, and nanoarrays. Solid supports also include mobile supports like beads.

### Kits

Further, disclosed herein are kits that are drawn to reagents that can be used in practicing the methods disclosed herein. The kits can include any reagent or combination of reagents discussed herein or that would be understood to be required or beneficial in the practice of the disclosed methods. For example, the kits could include one or more of the antibodies to alpha-synuclein disclosed herein, as well as the buffers, labels, enzymes, secondary or tertiary antibodies, etc. required to use the antibodies as intended.

### Methods of Making

The compositions (e.g., antibodies) disclosed herein and the compositions necessary to perform the disclosed methods can be made using any method known to those of skill in the art for that particular reagent or compound unless otherwise specifically noted.

In one aspect, one method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant, Synthetic Peptides: A User Guide. W.H. Freeman and Co., New York (1992); Bodansky and Trost, Ed. Principles of Peptide Synthesis. Springer-Verlag Inc., New York, (1993), which are herein incorporated by reference at least for material related to peptide synthesis.)

Independent peptides or polypeptides can be linked, if needed, to form a peptide or fragment thereof via similar peptide condensation reactions. For example, enzymatic ligation of cloned or synthetic peptide segments allow relatively short peptide fragments to be joined to produce larger peptide fragments, polypeptides or whole protein domains (Abrahmsen, et al., Biochemistry 30:4151 (1991)). Alternatively, native chemical ligation of synthetic peptides can be utilized to synthetically construct large peptides or polypeptides from shorter peptide fragments. This method comprises a two step chemical reaction (Dawson, et al., Science 266:776-9 (1994)). The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini, et al., FEBS Lett 307:97-101 (1992); Clark-Lewis, et al., J Biol Chem 269:16075 (1994); Clark-Lewis, et al., Biochemistry 30:3128 (1991); Rajarathnam, et al., Biochemistry 33:6623-30 (1994)). These references are incorporated by reference herein at least for their teachings of antibody preparation.

Alternatively, unprotected peptide segments are chemically linked where the bond formed between the peptide segments as a result of the chemical ligation is an unnatural (non-peptide) bond (Schnolzer, et al., Science 256:221 (1992)). This technique has been used to synthesize analogs of protein domains as well as large amounts of relatively pure proteins with full biological activity (deLisle Milton, et al., (1992) Techniques in Protein Chemistry IV. Academic Press, N.Y., pp. 257-67).

### Method of Using the Compositions

In one aspect, the antibodies disclosed herein can be used as reagents in a diagnostic assay for a neurodegenerative disease such as Parkinson's disease. For example, disclosed herein is a diagnostic assay for Parkinson's disease that comprises contacting a sample comprising a leukocyte or a lysate thereof with one or more antibodies or fragments thereof specific for alpha-synuclein. The sample can be as described above (e.g., a blood sample).

In another aspect, disclosed herein is a diagnostic assay for a neurodegenerative disease such Parkinson's disease that comprises contacting a sample from a subject to be diagnosed with an antibody as disclosed herein. In one example, the antibody can specifically bind a native monomeric alpha-synuclein. In another example, the antibody can specifically bind an alpha-synuclein bonded to dopamine quinone. In yet another example, the antibody can specifically bind an oligomeric or aggregated alpha-synuclein. In still another example, one or more antibodies that can specifically identify one or more alpha-synuclein forms can be use. For example, antibodies that specifically bind to the native monomer of alpha-synuclein and the dopamine-quinone bonded alpha-synuclein can be used, antibodies that specifically bind to the native monomer of alpha-synuclein and the oligomeric or aggregate form of alpha-synuclein can be used, antibodies that specifically bind to the oligomeric or aggregate forms of alpha-synuclein and the dopamine-quinone bonded alpha-synuclein can be used, and antibodies that specifically bind to the native monomer of alpha-synuclein, the dopamine-quinone bonded alpha-synuclein, and oligomeric or aggregate forms of alpha-synuclein can be used.

Method of Diagnosing Parkinson's Disease

In still another aspect, disclosed herein are methods of diagnosing Parkinson's disease in a subject. By “diagnose” or other forms of the word such as “diagnosing” and “diagnosis” is meant to identify a particular disease. The term also means to distinguish one particular disease from another disease or to distinguish one particular disease from the absence of disease. “Diagnose” is also used herein to mean to identify a particular stage of a disease, to identify the risk of developing a disease, or to identify a prognosis of a disease.

The disclosed methods comprise assessing a level of one or more alpha-synuclein conformers in a sample from the subject to be diagnosed and comparing the level of the alpha-synuclein conformer(s) to a reference standard that indicates the level of alpha-synuclein conformer(s) in one or more control subjects. In the disclosed methods a difference or similarity between the level of alpha-synuclein conformer(s) and the reference standard can indicate that the subject has Parkinson's disease.

In these particular methods, the subject can be as described herein, for example, any individual, such as a human. In one example, the subject is to be diagnosed for Parkinson's disease. The subject to be diagnosed can have symptoms of Parkinson's disease or the subject can be asymptomatic or preclinical for Parkinson's disease.

Assessing Levels of Expression

In the disclosed methods, the level of alpha-synuclein can be selected as the level of alpha-synuclein in the native monomer form, the level of alpha-synuclein bonded to dopamine-quinone, or the level of alpha-synuclein in the oligomeric or aggregate form, including any mixture or combination thereof. That is, in the disclosed methods, the level of expression of any single form of alpha-synuclein can be assessed. Also, the level of two or more forms of alpha-synuclein can be assessed. The levels of any single form of alpha-synuclein or the relative levels of two or more forms can be used to diagnose a subject with Parkinson's disease.

The assay can involve multiplex detection of multiple alpha-synuclein conformers. The results can be evaluated as a panel of markers. In one aspect, the relative amount of a conformers as compared to each of the other conformers is evaluated. One of skill in the art can chart the expression or relative expression of each conformer over the progression of the disease and create an association. This associate can then be used in the provided assays for diagnosis or prognosis.

In the disclosed methods, assessing a level of expression of alpha-synuclein in a sample can be performed by various techniques known in the art. For example, assessing the level of expression can involve analyzing one or more proteins by two-dimensional gel electrophoresis, mass spectroscopy (MS), matrix-assisted laser desorption/ionization-time of flight-MS (MALDI-TOF), surface-enhanced laser desorption ionization-time of flight (SELDI-TOF), high performance liquid chromatography (HPLC), fast protein liquid chromatography (FPLC), multidimensional liquid chromatography (LC) followed by tandem mass spectrometry (MS/MS), protein chip expression analysis, gene chip expression analysis, and laser densitometry, including combinations of these techniques. In another example of a technique for analyzing protein expression levels, one can assay the amount of mRNA that encodes for alpha-synuclein.

In another example, the antibodies disclosed herein, which selectively bind to alpha-synuclein conformers can be used to detect the amount of alpha-synuclein expressed in a sample. For example, the level of expression of alpha-synuclein can be measured using methods that include, but are not limited to, Western blot, immunoprecipitation, enzyme-linked immunosorbent assay (ELISA), radioimmunoassay (RIA), fluorescent activated cell sorting (FACS), or a combination thereof. Also, antibodies, aptamers, or other ligands that specifically bind to alpha-synuclein can be affixed to chips or microarrays and used to measure the level of expression of a specific conformer(s) in a sample. In other methods, immunofluorescence techniques can be used to visually assess the level of alpha-synuclein conformer(s) in a sample. In immunofluorescence techniques, antibodies that specifically bind to alpha-synuclein conformer(s) are visualized to indirectly detect the presence of a conformer(s) on the cell surface of intact cells, to detect the intracellular presence of a conformer(s), or to detect the presence of a conformer(s) in extracellular fluids (e.g., plasma, urine, etc).

Non-antibody ligands that selectively bind to alpha-synuclein conformer(s) can also be used to detect the presence, the absence, and/or to quantify the level of alpha-synuclein conformer(s). For example, ligands can be fluorescently labeled (e.g. conjugated to fluorescent molecule, such as green fluorescent protein (GFP)) or ligands can be radiolabeled. Labeled ligands can be contacted with a sample, and binding of the ligand to alpha-synuclein can be assessed. The amount of labeled ligand that binds to alpha-synuclein in the sample is an indication of the amount of alpha-synuclein present in the sample. Labels can be directly or indirectly attached to antibodies or non-antibody ligands. Direct labeling includes, for example, attaching a label directly to the antibody or non-antibody ligand. Indirect labeling includes, for example, attaching a label to a second or third antibody or non-antibody ligand.

Optionally, the level of expression of multiple conformers of alpha-synuclein can be determined simultaneously or nearly simultaneously. In this way, one can detect, for example, the levels of the native monomer form, the dopamine-quinone bonded form, and/or the oligomeric or aggregate form. For example, two-dimensional (2D) gel electrophoresis can be used to simultaneously or nearly simultaneously assess the expression level of thousands of proteins in a sample. (See, e.g., Vietor and Huber, Biochim Biophys Acta 1359:187-99 (1997), which is incorporated by reference herein for at least its teachings of methods to assess levels of protein expression). In one aspect, the disclosed methods can include 2D gel electrophoresis, where a mixture of proteins are prepared from the sample, e.g., by lysing cells and mixing the protein lysate with sample buffer. The protein mixture can be loaded onto a gel slab, electrophoresed in two dimensions, and then the gel slab can be dried. After resolution by 2D electrophoresis, expression levels of individual proteins or groups of proteins can be assessed. Protein levels can be assessed by silver staining or Coomassie staining. If the proteins in a sample are labeled, then measuring the amount of label can be used to assess the amount of protein.

In one aspect of the disclosed methods, the level of alpha-synuclein conformer(s) can be from about from about 15% to about 50%, from about 20% to about 45%, from about 25% to about 40%, or about 30% increase above normal levels. In other examples, the level of alpha-synuclein conformer(s) can be at least about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% above normal level, where any of the stated values can form an upper or lower endpoint when appropriate. Evidence from yeast studies indicates that about a 30% increase in alpha-synuclein is enough to induce toxicity (Dixon, et al., Alpha-Synuclein Targets the Plasma Membrane via the Secretory Pathway and Induces Toxicity in Yeast, Genetics E-published Mar. 21, 2005.

Comparing Levels of Expression

In the disclosed methods, when the level of alpha-synuclein conformer(s) is assessed, it can be compared with the levels in a reference standard. By “reference standard” is meant the level of alpha-synuclein in one or more control subjects. The control subject can be a subject that has a known condition, for example, the control subject can be a subject with Parkinson's disease, at a particular stage of a Parkinson's disease, with a particular risk of developing Parkinson's disease, without Parkinson's disease, or in the absence of a particular variable such as a therapeutic agent. The reference standard can also include the expression level of alpha-synuclein from one or more different samples or subjects as described herein (e.g., an average from several control subjects). Alternatively, a reference standard can be an average expression level of alpha-synuclein calculated from a number of subjects with or without Parkinson's disease. A reference standard can also include a known control level or value known in the art. In one aspect of the methods disclosed herein, it can be desirable to age-match and/or sex-match a reference standard with the subject diagnosed with Parkinson's disease. Thus, by comparing the level of alpha-synuclein from a subject to be diagnosed to the level of expression of alpha-synuclein in a control subject (i.e., reference standard), one can diagnose the subject for Parkinson's disease.

A difference or similarity in the level of alpha-synuclein expression can be determined by any quantitative or qualitative comparative analysis between the levels of expression of alpha-synuclein in the sample and in the reference standard. For example, when the control subject has Parkinson's disease, then when using the disclosed methods, a similarity between the level of alpha-synuclein expression in the subject and the control subject can indicate that the subject to be diagnosed also has Parkinson's disease. In another example, when the control subject has Parkinson's disease, then when using the disclosed methods, a difference between the level of alpha-synuclein expression in the subject and the control subject can indicate that the subject to be diagnosed does not have Parkinson's disease. Alternatively, when the control subject does not have a Parkinson's disease, then, in this example, a difference between the level of alpha-synuclein expression and the control subject can indicate that the subject to be diagnosed has Parkinson's disease. In still another example, when the control subject does not have Parkinson's disease, then, in this example, a similarity between the level of alpha-synuclein expression and the control subject can indicate that the subject to be diagnosed does not have Parkinson's disease.

In one technique to compare alpha-synuclein levels of expression from two different samples (e.g., a sample from a subject to be diagnosed with Parkinson's disease and a reference standard), each sample can be separately subjected to 2D gel electrophoresis. Alternatively, each sample can be differently labeled and both samples can be loaded onto the same 2D gel. See, e.g., Unlu, et al., Electrophoresis 18:2071-7 (1997), which is incorporated by reference herein for at least its teachings of methods to assess and compare levels of protein expression. Alpha-synuclein in each sample can be identified by the relative position within the pattern of proteins resolved by 2D electrophoresis. The expression levels of alpha-synuclein in a first sample can then be compared to the expression level of alpha-synuclein in the second sample.

Other methods can be used instead of 2D electrophoresis to identify the level of alpha-synuclein expression in a sample and compare that level to a reference standard, and can be used in the methods disclosed herein. Some of these methods utilize spectroscopic techniques such as surface-enhanced laser desorption ionization-time of flight (SELDI-TOF). Other methods rely on chromatographic techniques such as high performance liquid chromatography (HPLC), or fast protein liquid chromatography (FPLC). Multidimensional liquid chromatography (LC) and tandem mass spectrometry (MS/MS) can separate and identify multiple peptides. See Link, et al., Nat Biotechnol (1999), 17:676-82. Additional chromatographic methods for identifying multiple proteins are described in U.S. Patent Application Serial No. 394980. In still other methods, protein chips (arrays of protein binding antibodies, ligands, or aptamers) can be used to identify proteins that are expressed differently in a sample than in a reference standard. See, e.g., Glokler and Angenendt, J. Chromatogr B Analyt Technol Biomed Life Sci 797:229-40 (2003). Further, in vitro binding assay, such as radioimmunoassay (RIA) or enzyme-linked immunoabsorbent assay (ELISA) can be used to assess and compare levels of expression. Such techniques and assays are known in the art, and are described further in Harlow and Lane “Antibodies, A Laboratory Manual” Cold Spring Harbor Publications, New York, (1988). These references are incorporated by reference herein at least for their teachings of methods to assess and compare protein expression levels.

Methods of Monitoring Parkinson's Disease Progression

In still another aspect, disclosed herein are methods of monitoring Parkinson's disease progression in a subject. The disclosed methods can comprise comparing a level of alpha-synuclein conformer(s) in a sample obtained from the subject at multiple time points.

Also, disclosed herein are methods of monitoring a response to Parkinson's disease treatment in a subject. The disclosed methods can comprise comparing a level of alpha-synuclein conformer(s) in a sample obtained from the subject at multiple time points during treatment of the subject.

In these methods, the subject can be as disclosed above (e.g., human). Also, the subject can be asymptomatic or preclinical for Parkinson's disease at one or more of the multiple time points. In another example, the subject has not received treatment for Parkinson's disease at one or more of the multiple time points. In still another example, the subject can have received treatment for Parkinson's disease at one or more of the multiple time points.

By “treatment” is meant any medical intervention that the subject received or undergoes for the purpose of curing, preventing, or alleviating the disease. Treatment can include, but is not limited to, pharmacological therapy (e.g., the administration of pharmaceuticals), nutritional therapy (e.g., the administration of vitamins, hormones, nutraceuticals, trace elements, or supplements, or the alteration of diet), physical therapy, surgical treatment, and the like. Optionally, the subject receives treatment for Parkinson's disease at one or more of the multiple time points. Optionally, the subject is treated with a neuroprotective agent at or before one of the multiple time points. Optionally, the subject is treated with a dopamine agonist (e.g., levodopa) at one or more of the multiple time points. In another specific example, the subject is treated with a neuroprotective agent at one or more of the multiple time points.

In the disclosed methods, when the reference standard includes a level of alpha-synuclein conformer(s) in a sample or subject in the absence of a therapeutic agent, the sample or subject can be the same sample or subject before or after treatment with a therapeutic agent or can be a different sample or subject in the absence of the therapeutic agent.

Examples of neuroprotective agents which can be used to treat a subject include, but are not limited to, an acetylcholinesterase inhibitor, a glutamatergic receptor antagonist, kinase inhibitors, HDAC inhibitors, anti-flammatory agents, divalproex sodium, or any combination thereof. Examples of other neuroprotective agents can include, but are not limited to, Obidoxime Chloride; Pralidoxime Chloride; Pralidoxime Iodide; Pralidoxime Mesylate, Alverinc Citrate; Anisotropine Methylbromide; Atropine; Atropine Oxide Hydrochloride; Atropine Sulfate; Belladonna; Benapryzine Hydrochloride; Benzetimide Hydrochloride; Benzilonium Bromide; Biperiden; Biperiden Hydrochloride; Biperiden Lactate; Clidinium Bromide; Cyclopentolate Hydrochloride; Dexetimide; Dicyclomine Hydrochloride; Dihexyverine Hydrochloride; Domazoline Fumarate; Elantrine; Elucaine; Ethybenztropine; Eucatropine Hydrochloride; Glycopyrrolate; Heteronium Bromide; Homatropine Hydrobromide; Homatropine Methylbromide; Hyoscyamine; Hyoscyamine Hydrobromide; Hyoscyamine Sulfate; Isopropamide Iodide; Mepenzolate Bromide; Methylatropine Nitrate; Metoquizine; Oxybutynin Chloride; Parapenzolate Bromide; Pentapiperium Methylsulfate; Phencarbamide; Poldine Methylsulfate; Proglumide; Propantheline Bromide; Propenzolate Hydrochloride; Scopolamine Hydrobromide; Tematropium Methylsulfate; Tiquinamide Hydrochloride; Tofenacin Hydrochloride; Toquizine; Triampyzine Sulfate; Trihexyphenidyl Hydrochloride; Tropicamide. Further examples include, but are not limited to, Albutoin; Ameltolide; Atolide; Buramate; Carbamazepine; Cinromide; Citenamide; Clonazepam; Cyheptamide; Dezinamide; Dimethadione; Divalproex Sodium; Eterobarb; Ethosuximide; Ethotoin; Flurazepam Hydrochloride; Fluzinamide; Fosphenyloin Sodium; Gabapentin; Ilepcimide; Lamotrigine; Magnesium Sulfate; Mephenyloin; Mephobarbital; Methetoin; Methsuximide; Milacemide Hydrochloride; Nabazenil; Nafimidone Hydrochloride; Nitrazepam; Phenacemide; Phenobarbital; Phenobarbital Sodium; Phensuximide; Phenyloin; Phenyloin Sodium; Primidone; Progabide; Ralitoline; Remacemide Hydrochloride; Ropizine; Sabeluzole; Stiripentol; Sulthiame; Thiopental Sodium; Tiletamine Hydrochloride; Topiramate; Trimethadione; Valproate Sodium; Valproic Acid; Vigabatrin; Zoniclezole Hydrochloride; Zonisamide. Still other examples of anti-inflammatory agents include, but are not limited to, Aldlofenac; Aldlometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Difialone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; or Zomepirac Sodium.

In the disclosed methods, the level of alpha-synuclein conformer(s) can be determined as disclosed herein. Further, the level of alpha-synuclein conformer(s) can be the level of alpha-synuclein in the native monomer form, alpha synuclein bonded to dopamine-quinone, or alpha-synuclein in the oligomeric or aggregate form, including any mixture or combination thereof. Thus, one can assess the level of any single form of alpha-synuclein or the relative amounts of two or more forms of alpha-synuclein.

In the disclosed methods, a level of alpha-synuclein conformer(s) at one point in time can be the same as the level assessed at another point in time. This can indicate that Parkinson's disease has not changed (e.g., the disease has not gotten worse or better). In another example, a level of alpha-synuclein conformer(s) at an earlier point in time can be more or less than the level at a later point in time. In another example, a level of alpha-synuclein conformer(s) at an earlier point in time is less than the level at a later point in time. In still another example, while not wishing to be bound by theory, it is believed that the dopamine modified form of alpha-synuclein will be liberated from dying dopaminergic neurons (i.e., levels of alpha-synuclein bonded to dopamine quinone will be lower at an earlier point in time than at a later point in time), thus signaling a phase of cell loss. This change indicates that Parkinson's disease is progressing or that drug toxicity is occurring.

Also, the level of alpha-synuclein conformer(s) can be correlated with a worsening or an improvement in one or more symptoms of Parkinson's disease in response to the treatment. For example, a level of alpha-synuclein conformer(s) at one point in time before treatment can be the same as the level assessed after treatment. This can indicate that the treatment is effective at least in preventing the further progression of Parkinson's disease in the subject. In another example, a level of alpha-synuclein conformer(s) at an earlier point in time during treatment can be the same as the level assessed at later point in time during treatment. This can also indicate that the treatment is at least effective in preventing the further progression of Parkinson's disease in the subject. Expression levels of alpha-synuclein conformer(s) that are different between a sample taken prior to treatment or at an earlier point in time during treatment and a sample taken at a later point in time during treatment or after treatment can indicate that the treatment is effective/not effective in treating Parkinson's disease.

The change in expression levels can differ among different alpha-synuclein conformers. For example, during Parkinson's disease progression or during treatment the level of alpha-synuclein native monomer can increase over time while the level of dopamine-quinone bonded alpha-synuclein and/or oligomeric alpha-synuclein can decrease over time. Alternatively, during Parkinson's disease progression or during treatment the level of alpha-synuclein native monomer can decrease over time while the level of dopamine-quinone bonded alpha-synuclein and/or oligomeric alpha-synuclein can increase over time. In another alternative, during Parkinson's disease progression or during treatment the level of alpha-synuclein dopamine-quinone bonded alpha-synuclein can increase/decrease while the level of alpha-synuclein native monomer and/or oligomeric alpha-synuclein can decrease/increase. In yet another alternative, during Parkinson's disease progression or during treatment the level of oligomeric alpha-synuclein can increase/decrease while the level of alpha-synuclein native monomer and/or dopamine-quinone bonded alpha-synuclein can decrease/increase. Such changes in the levels of alpha-synuclein conformers can indicate that Parkinson's disease is progressing, at a particular stage, that a treatment is effective or not effective, that a treatment is toxic, and the like.

In these methods, a difference in a level of alpha-synuclein conformer(s) between various samples can be indicative of the subject's responsiveness to the administered treatment for the Parkinson's disease. If alpha-synuclein conformer(s)' expression has been previously shown to increase in subjects that (a) respond or (b) fail to respond to the treatment for a Parkinson's disease, then a larger amount of alpha-synuclein conformer(s) in a later sample relative to an earlier sample can be an indication that the subject is (a) responding or (b) not responding, respectively, to the treatment. Alternatively, if alpha-synuclein conformer(s)' expression has been previously shown to decrease in subjects that (a) respond or (b) fail to respond to a treatment for a Parkinson's disease, then a smaller amount of alpha-synuclein in a later sample relative to an earlier sample can be considered to be an indication that the subject is (a) responding or (b) not responding, respectively to the treatment.

Method of Treating or Preventing Parkinson's Disease

In still another aspect, disclosed herein are methods of treating or preventing Parkinson's disease in a subject. For example, provided is a method of treating Parkinson's disease in a subject, comprising administering to the subject a nucleic acid encoding a single chained antibody (scFv) that specifically binds alpha-synuclein. In one aspect, the scFv can specifically bind all conformers of alpha-synuclein. In another aspect, the scFv can specifically bind one or more alpha-synuclein conformers. Thus, the scFv can specifically bind alpha-synuclein monomer. The scFv can specifically bind alpha-synuclein bonded to dopamine-quinone. The scFv can specifically bind alpha-synuclein in the oligomeric or aggregate form. The scFv can specifically bind alpha-synuclein monomer and alpha-synuclein in the oligomeric or aggregate form but not alpha-synuclein bonded to dopamine-quinone. Other such combinations of conformer epitopes are considered and disclosed herein.

The herein disclosed antibodies can be administered to a subject for the purpose of immunotherapy. In one aspect, the antibodies are administered as nucleic acids that encode said antibodies. For example, provided is a nucleic acid encoding an scFv specific for a SYN conformer. Further, also provided is a vector comprising said scFv-encoding nucleic acid. The vector can be any vector capable of delivering a nucleic acid to the brain, including, but not limited to viral vectors. In a preferred embodiment, the vector is an AAV vector.

Nucleic Acid Based Delivery of Antibodies Such as scFv

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as those encoding scFvs into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by lipo some-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19.

Adeno-associated virus (AAV) is a member of the Parvoviridae, a virus family characterized by a single stranded linear DNA genome and a small icosahedral shaped capsid measuring about 20 nm in diameter. AAV was first described as a contamination of tissue culture grown simian virus 15, a simian adenovirus and was found dependent on adenovirus for measurable replication. This lead to its name, adeno-associated virus, and its classification in the genus Dependovirus (reviewed in Hoggan, M. D. Prog Med Virol 12 (1970) 211-39). AAV is a common contaminant of adenovirus samples and has been isolated from human virus samples (AAV2, AAV3, AAV5), from samples of simian virus-15 infected cells (AAV1, AAV4) as well as from stocks of avian (AAAV) (Bossis, I. and Chiorini, J. A. J Virol 77 (2003) 6799-810), bovine, canine and ovine adenovirus and laboratory adenovirus type 5 stock (AAV6). DNA spanning the entire rep-cap ORFs of AAV7 and AAV8 was amplified by PCR from heart tissue of rhesus monkeys (Gao, G. P., et al. Proc Natl Acad Sci USA 99 (2002) 11854-9). With the exception of AAVs 1 and 6, all cloned AAV isolates appear to be serologically distinct. Nine isolates have been cloned, and recombinant viral stocks have been generated from each isolated virus.

AAV2 is the best characterized adeno-associated virus and will be discussed as an AAV prototype. The AAV2 genome consists of a linear single stranded DNA of 4,780 nucleotides. Both polarities of DNA are encapsulated by AAV with equal efficiency. The AAV2 genome contains 2 open reading frames (ORF) named rep and cap. The rep ORF encodes the non-structural proteins that are essential for viral DNA replication, packaging and AAV integration. The cap ORF encodes the capsid proteins. The rep ORF is transcribed from promoters at map units P5 and P19. The rep transcripts contain an intron close to the 3′ end of the rep ORF and can be alternatively spliced. The rep ORF is therefore expressed as 4 partially overlapping proteins, which were termed according to their molecular weight Rep78, 68, 52 and 40. The cap ORF is expressed from a single promoter at P40. By alternative splicing and utilization of an alternative ACG start codon, cap is expressed into the capsid proteins VP1-3 which range in size from 65-86 kDa. VP3 is the most abundant capsid protein and constitutes 80% of the AAV2 capsid. All viral transcripts terminate at a polyA signal at map unit 96.

During a productive AAV2 infection, unspliced mRNAs from the p5 promoter encoding Rep78 are the first detectable viral transcripts. In the course of infection, expression from P5, P19 and P40 increase to 1:3:18 levels respectively. The levels of spliced transcripts increased to 50% for P5, P19 products and 90% of P40 expressed RNA (Mouw, M. B. and Pintel, D. J. J Virol 74 (2000) 9878-88).

The AAV2 genome is terminated on both sides by inverted terminal repeats (ITRs) of 145 nucleotides (nt). 125 nt of the ITR constitute a palindrome which contains 2 internal palindromes of 21 nt each. The ITR can fold back on itself to generate a T-shaped hairpin with only 7 non-paired bases. The stem of the ITR contains a Rep binding site (RBS) and a sequence that is site and strand specifically cleaved by Rep—the terminal resolution site (TRS). The ITR is essential for AAV2 genome replication, integration and contains the packaging signals.

The single-stranded AAV2 genome is packaged into a non-enveloped icosahedral shaped capsid of about 20-25 nm diameter. The virion consists of 26% DNA and 74% protein and has a density of 1.41 g/cm3. AAV2 particles are extremely stable and can withstand heating to 60° C. for 1 hour, extreme ph, and extraction with organic solvents.

Rep proteins are involved in almost every step of AAV2 replication including AAV2 genome replication, integration, and packaging. Rep78 and Rep68 possess ATPase, 3′-5′ helicase, ligase and nicking activities and bind specifically to DNA. Rep52 and Rep40 appear to be involved in the encapsidation process and encode ATPase and 3′-5′ helicase activities. Mutational analysis suggests a domain structure for Rep78. The N-terminal 225 aa are involved in DNA binding, DNA nicking and ligation. Rep78 and Rep68 recognize a GCTC repeat motif in the ITR as well as in a linear truncated form of the ITR (Chiorini, J. A., et al. J Virol 68 (1994) 7448-57) with similar efficiencies. Rep78 and Rep68 possess a sequence and strand specific endonuclease activity, which cleaves the ITR at the terminal resolution site (TRS). Rep endonuclease activity is dependent on nucleoside triphosphate hydrolysis and presence of metal cations. Rep 78 and 68 can also bind and cleave single stranded DNA in a NTP independent matter. In addition, Rep78 catalyzes rejoining of single stranded DNA substrates originating from the AAV2 origin of replication—i.e., sequences containing a rep binding and terminal resolution element.

The central region of AAV2 Rep78, which represents the N-terminus of Rep52 and Rep40, contains the ATPase and 3′-5′ helicase activities as well as nuclear localization signals. The helicase activity unwinds DNA-DNA and DNA-RNA duplexes, but not RNA-RNA. The ATPase activity is constitutive and independent of a DNA substrate. The C-terminus of Rep78 contains a potential zinc-finger domain and can inhibit the cellular serine/threonine kinase activity of PKA as well as its homolog PRKX by pseudosubstrate inhibition. Rep68 which is translated from a spliced mRNA that encodes the N-terminal 529 amino acids (aa) of Rep78 fused to 7 aa unique for Rep68, doesn't inhibit either PKA or PRKX. In addition to these biochemical activities, Rep can affect intracellular conditions by protein-protein interactions. Rep78 binds to a variety of cellular proteins including transcription factors like SP-1, high-mobility-group non-histone protein 1 (HMG-1) and the oncosuppressor p53. Overexpression of Rep results in pleiotrophic effects. Rep78 disrupts cell cycle progression and inhibits transformation by cellular and viral oncogenes. In susceptible cell lines, overexpression of Rep resulted in apoptosis and cell death. Several of Rep78 activities contribute to cytotoxicity, including its constitutive ATPase activity, interference with cellular gene expression and protein interactions.

The first step of an AAV infection is binding to the cell surface. Receptors and coreceptors for AAV2 include heparan sulfate proteoglycan, fibroblast growth factor receptor-1, and αvβ5 integrins whereas N-linked 2,3-linked sialic acid is required for AAV5 binding and transduction (Walters, R. W., et al. J Biol Chem 276 (2001) 20610-6). In HeLa cells, fluorescently labeled AAV2 particles appear to enter the cell via receptor-mediated endocytosis in clathrin coated pits. More than 60% of bound virus was internalized within 10 min after infection. Labeled AAV particles are observed to have escaped from the endosome, been trafficked via the cytoplasm to the cell nucleus and accumulated perinuclear, before entering the nucleus, probably via nuclear pore complex (NPC). AAV2 particles have been detected in the nucleus, suggesting that uncoating takes place in the nucleus (Bartlett, et al. J Virol 74 (2000) 2777-85; Sanlioglu et al. J Virol 74 (2000) 9184-96). AAV5 is internalized in HeLa cells predominantly by clathrin coated vesicles, but to a lesser degree also in noncoated pits. AAV particles can also be trafficked intercellularly via the Golgi apparatus (Bantel-Schaal, U., et al. J Virol 76 (2002) 2340-9). At least partial uncoating of AAV5 was suggested to take place before entering the nucleus since intact AAV5 particles could not be detected in the nucleus (Bantel-Schaal et al., 2002) After uncoating, the single stranded genome is converted into duplex DNA either by leading strand synthesis or annealing of input DNA of opposite polarity. AAV replication takes place within the nucleus.

During a co-infection with a helper virus such as Adenovirus, herpes simplex virus or cytomegalovirus, AAV is capable of an efficient productive replication. The helper functions provided by Adenovirus have been studied in great detail. In human embryonic kidney 293 cells, which constitutively express the Adenovirus E1A and E1B genes, the early Adenovirus gene products of E2A, E4 and VA were found sufficient to allow replication of recombinant AAV. Allen et al. reported that efficient production of rAAV is possible in 293 cells transfected with only an E4orf6 expression plasmid (Allen, J. M., et al. Mol Ther 1 (2000) 88-95). E1A stimulates S phase entry and induces unscheduled DNA synthesis by inactivating the pRB checkpoint at the G1/S border by interaction with pRB family proteins which results in the release of E2F (reviewed in (Ben-Israel, H. and Kleinberger, T. Front Biosci 7 (2002) D1369-95). This leads to either induction or activation of enzymes involved in nucleotide synthesis and DNA replication. Since unscheduled DNA synthesis is a strong apoptotic signal, anti-apoptotic functions are required. E1B-19k is a Bcl-2 homolog and E1B-55k is a p53 antagonist. Both proteins have anti-apoptotic functions. E4orf6 forms a complex with E1B-55k and results in degradation of p53. It is also reported to cause S-phase arrest (Ben-Israel and Kleinberger, 2002). E2A encodes a single strand DNA binding protein, which appears to be non-essential for DNA replication but effects gene expression (Chang and Shenk. J Virol 64 (1990) 2103-9). The VA transcription unit affects AAV2 RNA stability and translation (Janik et al., Virology 168 (1989) 320-9). E1A has a more direct effect on AAV2 gene expression. The cellular transcription factor YY-1 binds and inhibits the viral P5 promoter. E1A relieves this transcriptional block. None of the late Ad gene products have been found to be essential for AAV2 replication. The main function of the helper virus appears to be the generation of a cellular environment with active DNA replication machinery and blocked pro-apoptotic functions that allows high-level AAV replication rather than a direct involvement in AAV replication.

Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed xxx or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR— cells and mouse LTK− cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

Pharmaceutical Carriers/Delivery

The disclosed compositions, such as antibodies or nucleic acids, can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

Screening of the human single chain antibody (scFv) library with monomeric alpha-synuclein identified two specific antibodies. Further screening with dopamine-adducted aggregated alpha-synuclein identified an additional eight antibodies (Table 1). Binding specificity of the scFv was determined by ELISA (Table 1). scFv's specific for monomeric alpha-synuclein, dopamine-adducted alpha-synuclein, and aggregated alpha-synuclein were identified. These scFv's can be used to screen human blood, urine, CSF for conformer specific alpha-synuclein.

TABLE 1
alpha-synuclein-specific scFv's

| Clone No. | Antigen panned against* | Antigen recognized** |
|---|---|---|
| 14 | alpha-synuclein monomer | alpha-synuclein monomer |
| 15 | alpha-synuclein monomer | alpha-synuclein monomer |
| 3 | alpha-synuclein:DAQ | alpha-synuclein monomer, |
| | | alpha-synuclein |
| | | aggregates, SYN:DAQ |
| 4 | alpha-synuclein:DAQ | SYN:DAQ, BSA:DAQ |
| 5 | alpha-synuclein:DAQ | alpha-synuclein monomer, |
| | | alpha-synuclein |
| | | aggregates, SYN:DAQ, |
| | | BSA:DAQ |
| 6 | alpha-synuclein:DAQ | SYN:DAQ, BSA:DAQ |
| 7 | alpha-synuclein:DAQ | SYN:DAQ, BSA:DAQ |
| 8 | alpha-synuclein:DAQ | SYN:DAQ, BSA:DAQ |
| 10 | alpha-synuclein:DAQ | SYN:DAQ, BSA:DAQ |

*Antigen panned against: a human scFv library was panned against different conformers of alpha-synuclein and phage identified.
**Antigen recognized: following initial panning scFv were expressed, purified, and tested against the full panel of alpha-synuclein conformers.

### Example 2

The linear peptide recognition site for three of the identified anti-synuclein scFvs were determined. Specifically, biotinylated 15-mer synthetic peptides spanning human alpha-synuclein were synthesized and plated onto strepavidin microtiter plates. ScFvs were incubated with the individual peptides and interactions detected using a microplate reader. The results are shown in Table 2.

TABLE 2

Results of linear alpha-synuclein peptide
mapping for anti-synuclein scFvs.

| Clone | Linear | | |
|---|---|---|---|
| # | Peptide | Peptide Sequence | SEQ ID NO |
| 14 | aa 106-120 | GAPQEGILEDMPVDP | SEQ ID NO:2 |
| 15 | aa 117-131 | MPVDPDNEAYEMPSE | SEQ ID NO:3 |
| `3 | aa 71-85 | VTGVTAVAQKTVEGA | SEQ ID NO:4 |

### Example 3

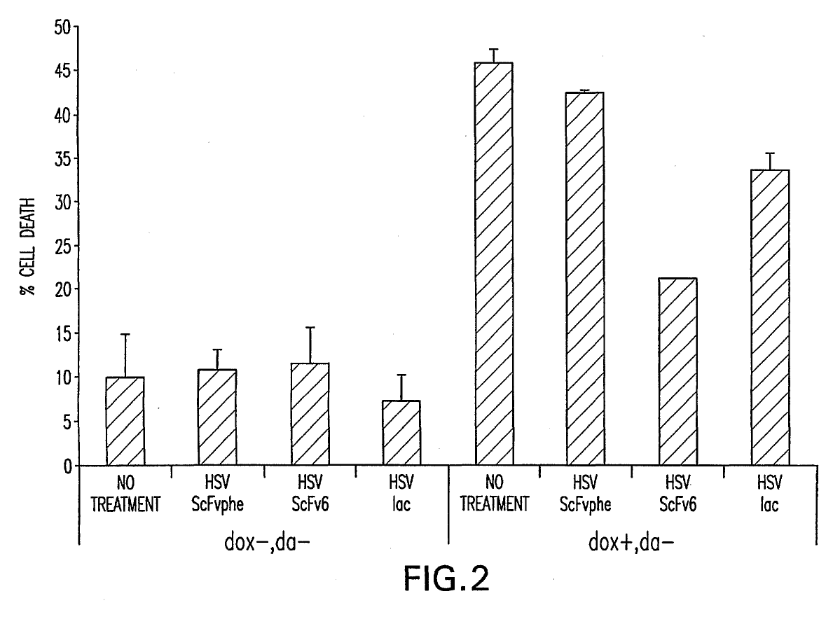

The ability of one scFv to inhibit cell death due to overexpression of alpha-synuclein in a dopaminergic cell line was examined. MN9Dαsyn cells were grown in the presence of doxycycline to induce alpha-synuclein expression. Overexpression of alpha-synuclein routinely causes cell death under these conditions as measured by flow cytometry following propidium iodide treatment (see FIG. 2; 45% cell death in presence of alpha-synuclein). As shown in FIG. 2, transduction of cells with an HSV amplicon expressing scFv6 attenuated the alpha-synuclein induced cell death (approximately 20% reduction). Amplicons expressing scFvphe (a scFv antibody which recognizes Phenobarbital) or HSVlac (beta-galactosidase) had no effect on alpha-synuclein induced cell death.

### Example 4

### Results

### Identification of Anti-Synuclein Single-Chain Antibodies

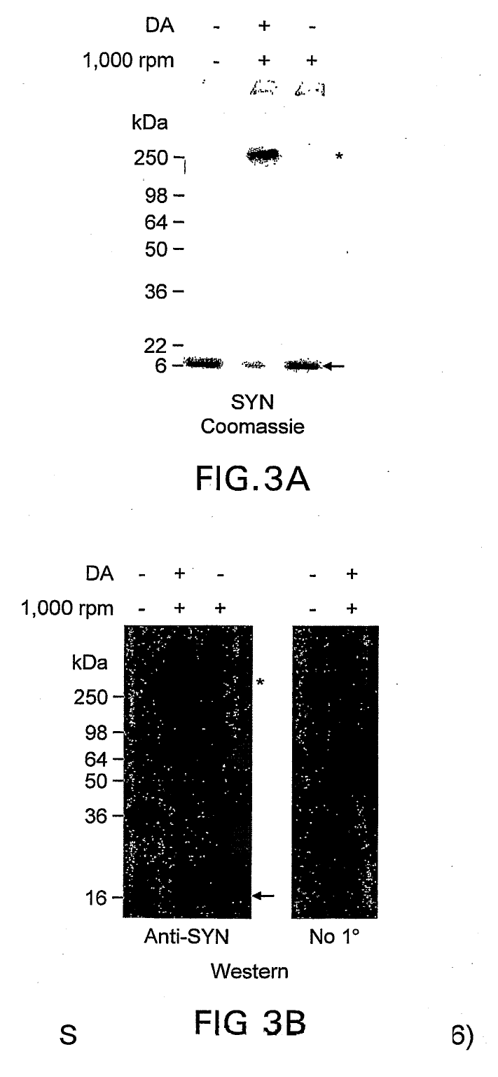

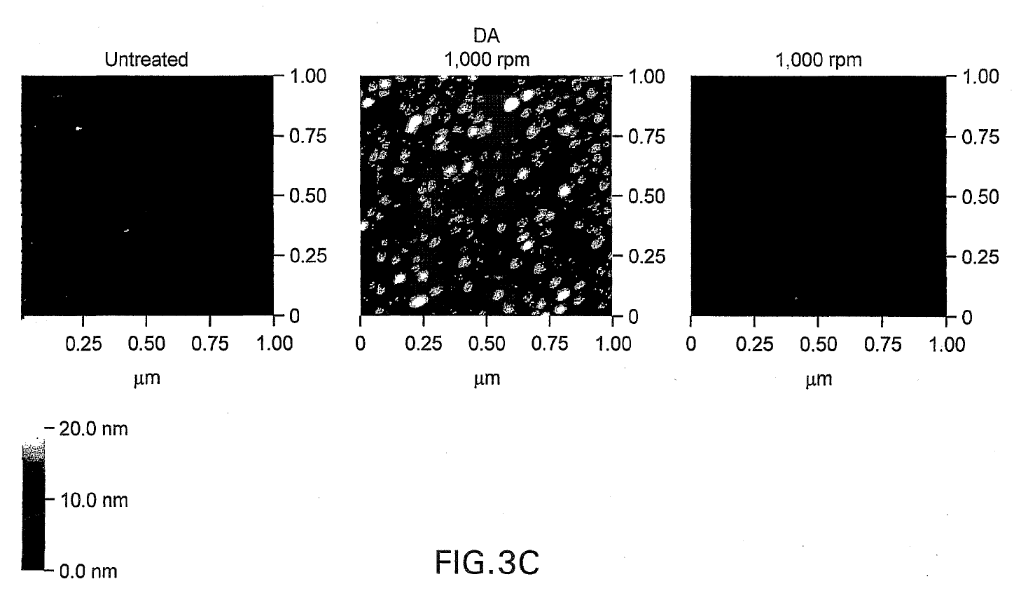

In its native form human wild-type α-synuclein (SYN) exists as a random coil but can misfold into oligomers and large molecular weight aggregates. Since misfolded SYN is toxic in both cell culture and animal models, human SYN was bacterially produced and biochemically purified, which was subsequently experimentally induced to misfold. both monomeric and misfolded SYN were then utilized to pan against a human phage library harboring single-chain antibodies (scFv). Specifically, monomeric SYN was expressed and biochemically purified from bacteria. Misfolded higher order SYN aggregates were formed following incubation of monomeric SYN at 33° C. with shaking (1,000 rpm) in both the absence and presence of dopamine (DA). As shown in FIG. 3A, following polyacrylamide gel electrophoresis under denaturing conditions (SDS-PAGE) and Coomassie blue staining, purified SYN appeared as a single 16 kDa band. Following incubation with DA/1,000 rpm, there was a loss of the 16 kDA protein and the concomitant appearance of large aggregates (+250 kDa) including protein that was unable to enter the resolving gel (*). Large aggregates are also apparent albeit to a lesser extent when SYN is incubated with agitation in the absence of DA. To improve the sensitivity of detection the SYN conformers were subjected to western blot analysis following SDS-PAGE (FIG. 3B). Again various SYN conformers were identified following modification with DA and/or 1,000 rpm agitation. Finally, SYN conformers were subjected to atomic force microscopy (AFM) which revealed a greater number of large SYN aggregates (10-20 nm; z plane) in the DA-treated SYN samples than either the monomeric SYN or SYN subjected to agitation (FIG. 3C).

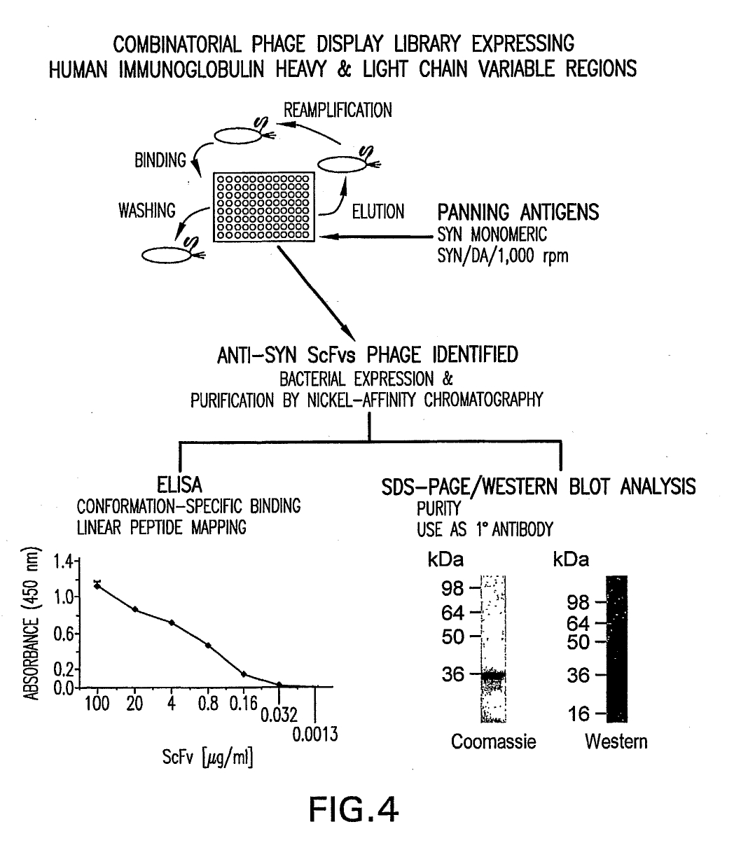

These characterized SYN conformers were than utilized to pan for SYN conformer-specific scFvs using a human phage display library (FIG. 4). The human single-chain antibody phage display library was generated in AP-III6 (Haidaris, C. G., et al., 2001), a phage display vector designed to promote stable, low level display of scFvs. The library was generated by PCR amplification of VL and VH immunoglobulin domains from human leukocyte cDNA prepared from >100 donors. The variable regions were amplified with PCR primers that encode a 14 amino acid linker between the VL and VH domains, and when cloned into pAP-III6, the scFvs contain an amino-terminal FLAG™ sequence (DYKDDDDK, SEQ ID NO:5). The library consists of approximately 2×10⁹ independent transformants. Three rounds of enrichment were carried out and the number of phage bound in the well was observed to have increased approximately 1,000 fold relative to the initial round of enrichment. At this point, individual colonies were picked, infected with helper, and the phage produced were tested for binding to SYN with a phage Enzyme-Linked Immunosorbent Assay (ELISA). Plasmid DNA from positive clones was prepared and gene III was removed to generate a clone secreting soluble scFv containing a carboxy-terminal polyhistidine tag. Alternatively, selected SYN scFvs were PCR subcloned into pComb3x. Identification and purification of scFvs was facilitated by virtue of C-terminal polyhistidine (His) and influenza hemagglutinin (HA) tags and an N-terminal FLAG™-tag. Using nickel-affinity chromatography the different scFvs were purified in sufficient quantities for further manipulations. Both Coomassie blue staining and western blot analysis of purified scFv preparations revealed the predicted 32 kDa protein. Purified scFvs were then subjected to an ELISA with different SYN conformers. Three groups of scFvs which recognize either monomeric SYN, aggregated SYN, DA modified SYN and/or DA modified bovine serum albumin were subsequently identified (Table 3).

TABLE 3
Antigens recognized by anti-SYN human ScFvs

| | | SYN:DA | BSA:DA | SYN |
|---|---|---|---|---|
| ScFv | SYN | 1,000 rpm | 1,000 rpm | 1,000 rpm |
| 14 | + | − | − | + |
| 15 | + | − | − | + |
| 3 | + | + | − | + |
| 5 | + | + | − | + |
| 4 | − | + | + | − |
| 6 | − | + | + | − |

### Characterization of Conformer-Specific Single-Chain Antibodies

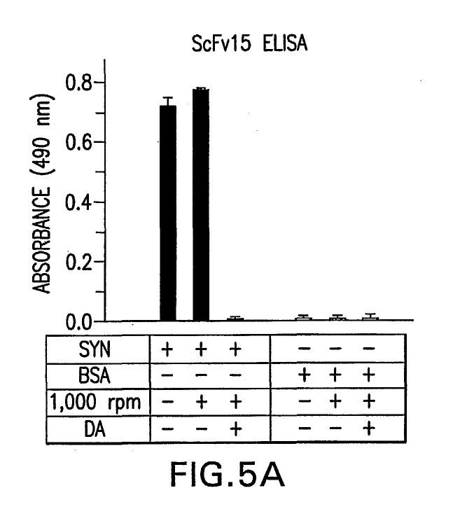

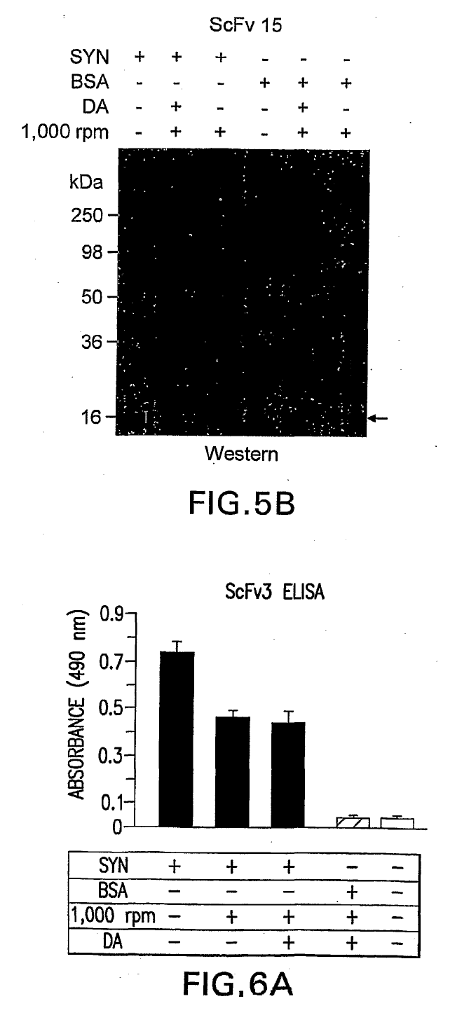

Once identified the conformer-specific scFvs were further characterized. Firstly, scFv15, which was identified following a phage panning with monomeric SYN, was studied. In an ELISA, scFv15 bound to native SYN and SYN aggregated at 1,000 rpm (SYN/1,000 rpm) but not DA modified SYN (SYN/DA/1,000 rpm) or a non-specific protein control (BSA; FIG. 5A). The ability of scFv15 to act as a primary antibody to identify SYN was further tested following polyacrylamide gel electrophoresis under denaturing conditions (SDS-PAGE) in a western blot analysis. Denatured monomeric SYN was readily detectable as a 16 kDa protein by scFv15 (FIG. 5B). Furthermore, scFv15 also recognized misfolded and aggregated SYN in both the absence and presence of DA (FIG. 5B). ScFv15 did not recognize similarly modified BSA.

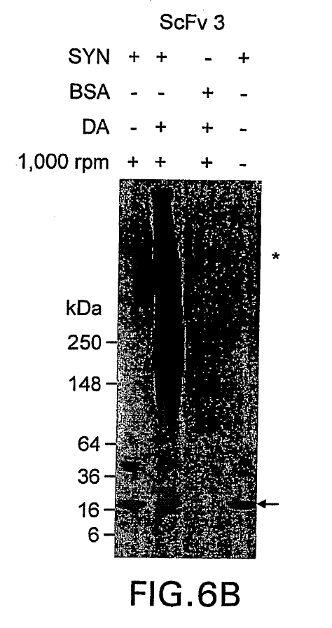

Secondly, scFv3 was evaluated and it was determined this scFv reacts with all SYN conformers in an ELISA (FIG. 6A). Similarly, under denaturing conditions, scFv3 recognizes monomeric, oligomeric and higher molecular weight SYN aggregates including those modified by DA (FIG. 6B). There is negligible scFv3 reactivity with DA-modified BSA.

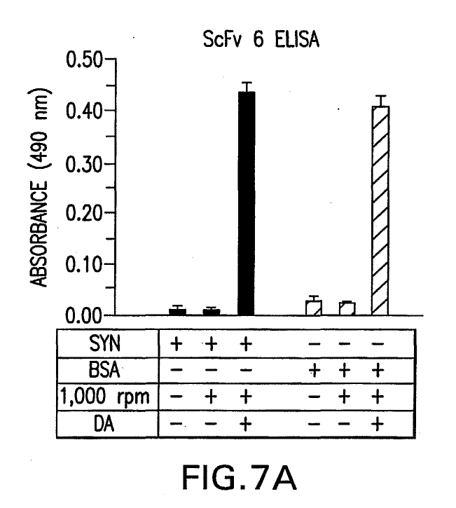

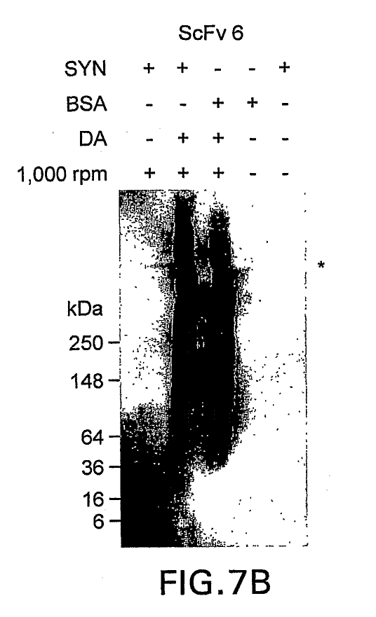

scFvs that had specific reactivity with DA-modified proteins were investigated. As demonstrated in FIG. 7, scFv6 recognizes DA-modified SYN and BSA (SYN:DA: 1,000 rpm; BSA:DA: 1,000 rpm) but did not interact with monomeric or aggregated SYN or BSA in the absence of DA. This scFv was also capable of identifying DA-modified proteins following denaturing polyacrylamide gel electrophoresis and western blot analysis.

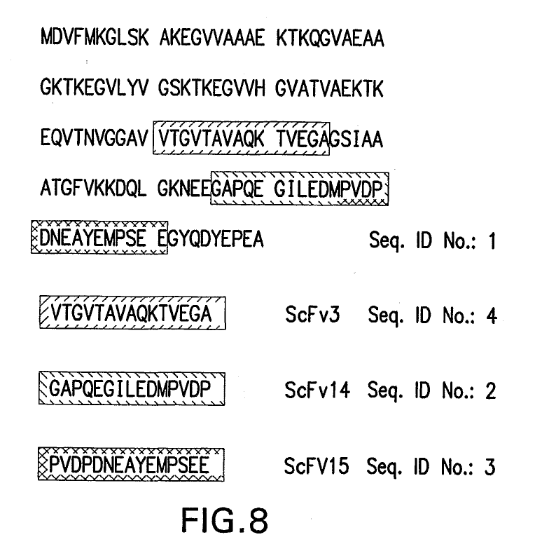

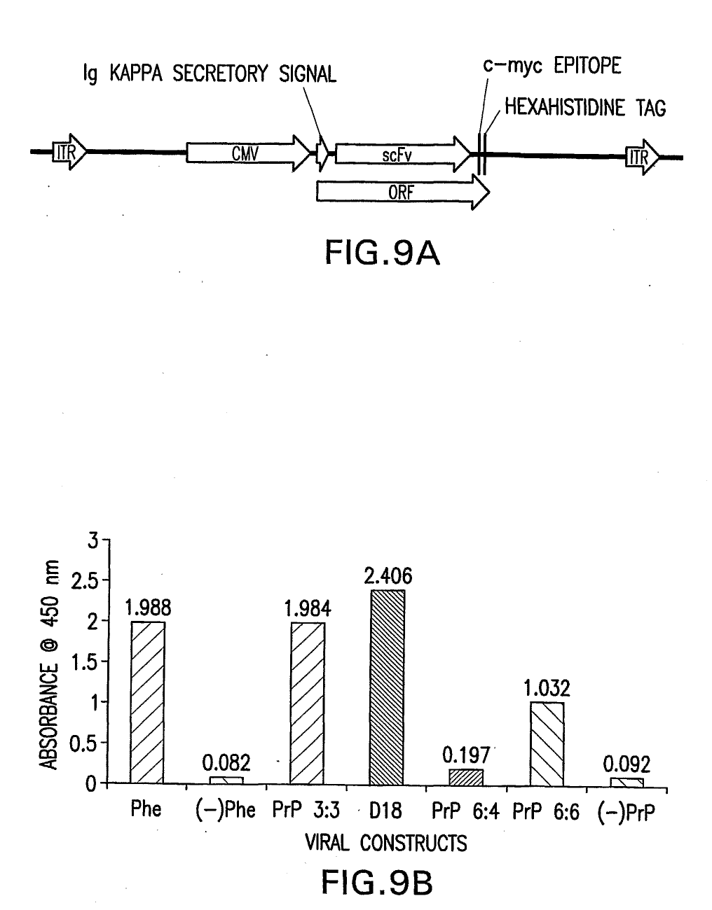

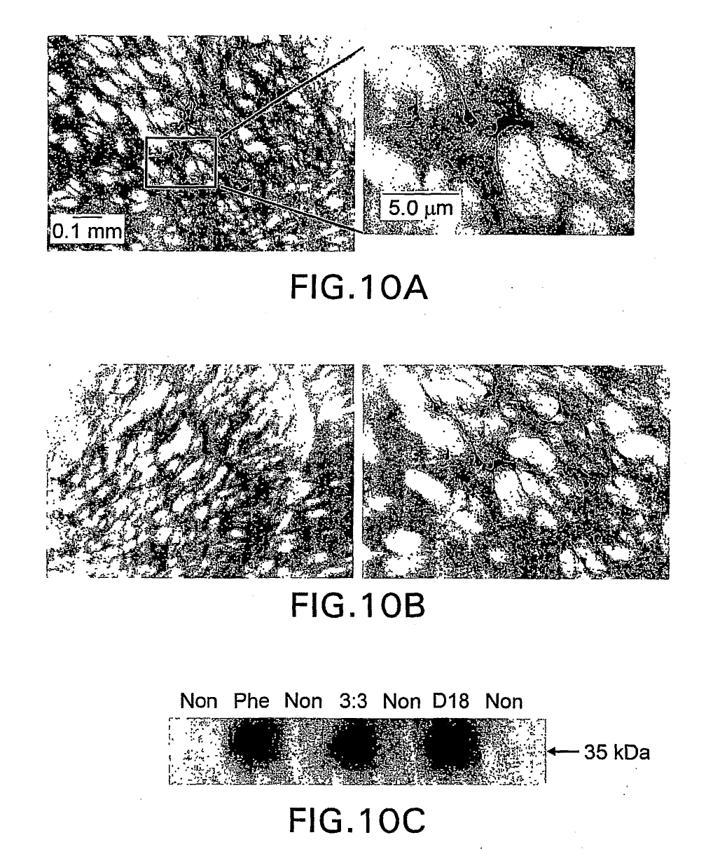

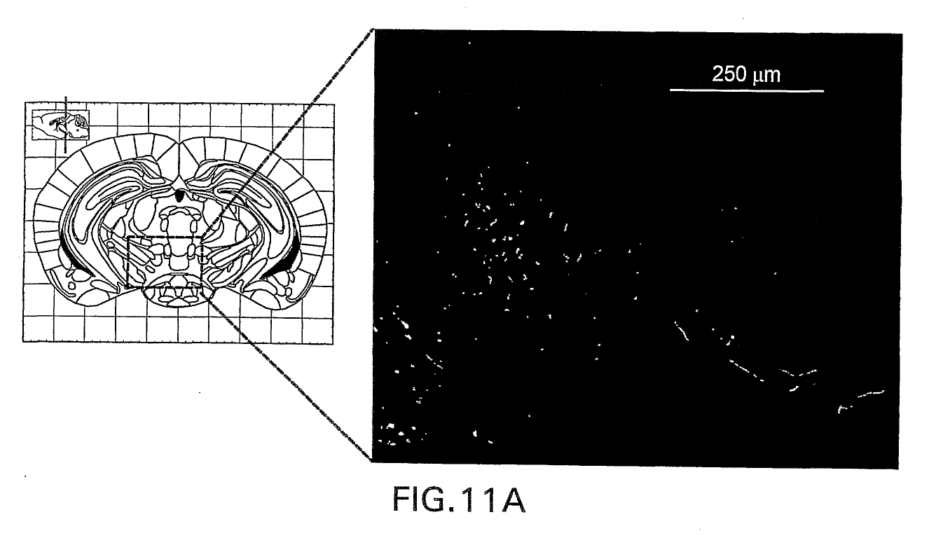

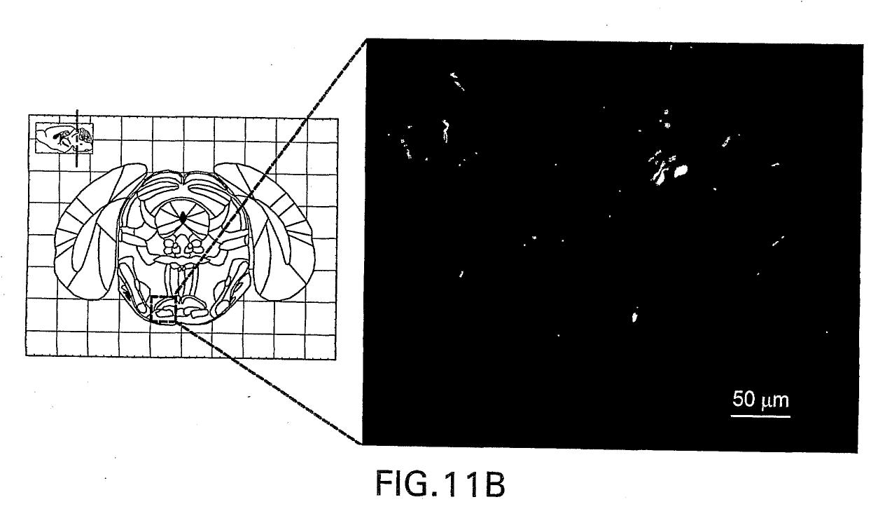

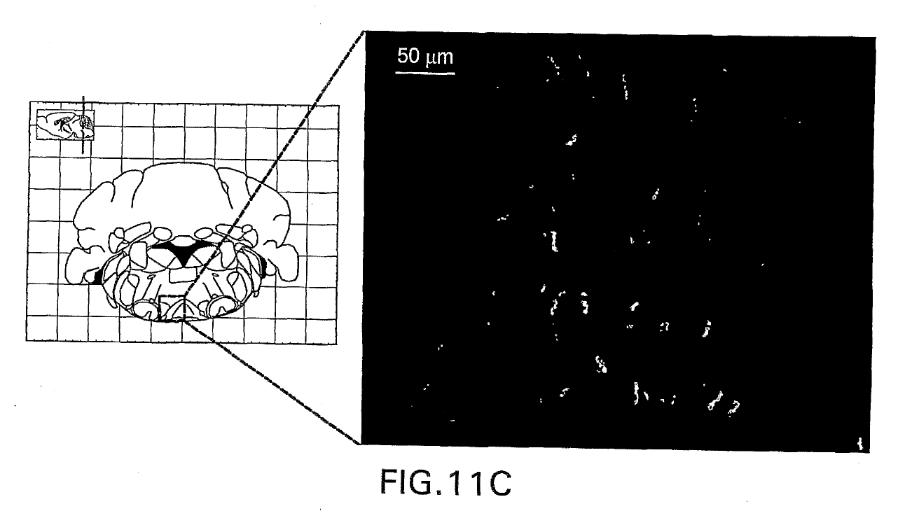

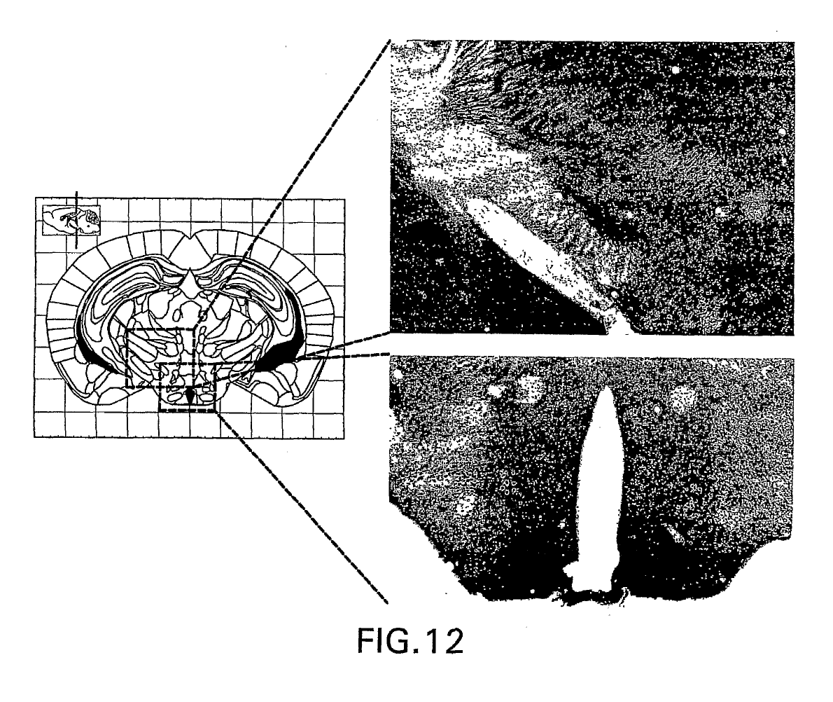

Since scFv14, 15 and 3 all recognized monomeric SYN, the SYN linear peptide recognition site was investigated. Using biotinylated 15 amino acid overlapping peptides spanning the entire SYN sequence attached to strepavidin-coated plates the specific binding sites for the three monomeric SYN recognizing scFvs were identified. As shown in FIG. 8, scFv14 recognized amino acids 106-120, scFv15 recognized amino acids 117-131 and scFv3 recognized amino acids 71-84. As expected, scFv6 which recognizes DA-modified SYN and BSA but not monomeric SYN did not bind SYN linear peptide sequences.

### Materials and Methods

Antibodies—Commercially available antibodies were utilized for synuclein and scFv detection (mouse anti-α-synuclein, BD Biosciences; mouse monoclonal anti-M2 FLAG™, Sigma). Horseradish peroxidase conjugated (HRP) secondary antibodies were from Amersham. In some cases primary antibodies were HRP-conjugated and used for direct detection (anti-M2 FLAG™-HRP, Sigma; anti-HA-HRP, Roche Diagnostics).

Bacterial Expression and Purification of α-Synuclein—The bacterial expression vector pRK172 containing wild-type human α-synuclein cDNA was provided (Giasson, B. I., et al., J Biol Chem, 1999). pRK172 α-synuclein was expressed and purified from Escherichia coli BL21 as described (Giasson, B. I., et al., J Biol Chem, 1999). Bacterial pellets were resuspended in high salt buffer (750 mM NaCl, 10 mM Tris-HCl, pH 7.5, 1 mM EDTA; 750 mM TEN) with protease inhibitors, boiled 10 minutes and supernatant recovered following centrifugation at 12,500 rpm for 15 minutes. Cleared supernatants were dialyzed against 20 mM TEN and applied to a Mono S column, unbound protein collected and immediately applied to a Mono Q column. SYN was eluted using a step gradient (100 mM, 250 mM, 500 mM and 2M TEN). For some preparations, cleared lysates were applied directly to a Mono Q column. Purification was confirmed by silver staining of proteins following SDS-PAG electrophoresis under denaturing conditions and western blot analysis (mouse anti-α-synuclein; BD Biosciences).

Treatment of α-Synuclein—One mg/ml of α-synuclein was incubated in the presence and absence of 3.5 mM (final concentration) dopamine at 33° C. with shaking (1,000 rpm) for various times as indicated in the figure legends.

Atomic Force Microscopy—Human wildtype α-synuclein was incubated in buffer containing 100 mM Tris-Cl pH 7.5, 1 mM EDTA and 20 mM NaCl at 33° C. in the presence or absence of 3.5 mM dopamine with gentle agitation (1000 rpm) for various times. Five μl of each sample was placed upon freshly cleaved mica discs (Ted Pella, Redding, Calif.), allowed to dry for 5 minutes and washed three times with 20 μl double distilled water. Images from the atomic force microscope were captured utilizing a conductive probe (Nanoscience Instruments, Phoenix, Ariz.) in tapping mode. Images were visualized with Nanoscope NT Offline Software (Version 5.12R4).

Selection of phage scFvs to SYN—Phage Library Panning. A human single-chain antibody phage display library was generated in AP-III6 (Haidaris, C. G., et al., 2001). After infection with helper phage (M13 VCS, Stratagene) and overnight growth at 30° C., the phage were concentrated by polyethylene glycol precipitation, resuspended in buffer containing 50 mM Tris, pH 8.0, 150 mM NaCl (TBS), 0.5% casein and 15% glycerol. Phage were subsequently stored at −80° C. Enrichment of phage libraries. General methods for phage display have been described (Barbas, C. F., 2001). Monomeric SYN or SYN/DA/1,000 rpm was coated in microtiter plate wells at 50 μg/ml in TBS overnight. After removal of the solution, the wells were blocked with TBS containing 0.5% casein. Aliquots of the phage library were applied to the wells and incubated for two hours with shaking. The phage were removed and the wells hand washed 7 times with a pipettor using TBS containing 0.5% Tween 20. After an additional water rinse, the phage were eluted from the wells by incubation with 0.1 M glycine HCl, pH 2.0 for 15 minutes and then neutralized with Tris base. The eluted phage were transduced into TG1 cells and plated on LB plates containing ampicillin. The next day, the colonies were scraped off, resuspended in LB medium, diluted to 1×10⁷ cells per ml, regrown and infected with helper phage to prepare a new pool of phage for biopanning. This procedure was repeated for a third round of enrichment. Individual colonies were picked, infected with helper, and the phage produced tested for binding to SYN with a phage ELISA assay, using anti-M13-horseradish peroxidase conjugate (GE Healthcare) to detect bound phage. Plasmid DNA from positive clones was prepared and gene III was removed by Sal I+Xho I digestion and re-ligation to generate a clone secreting soluble scFv containing a carboxy-terminal His tag. Alternatively, selected SYN scFvs were PCR subcloned into pComb3x appending a hexahistidine sequence to the 3′ end and a FLAG™ sequence to the 5′ end of the scFv to facilitate purification of the expressed protein. Distinct binders were identified by BstNI fingerprinting of the PCR-amplified scFvs and sequenced.

Expression and purification of scFvs—ScFvs were expressed either by growth of cultures in a low phosphate containing medium (Simmons, L. C., et al., 2002) to induce expression from the phoA promoter of the display vector or expressed in E. coli following IPTG induction of the cultures. Expressed his-tagged scFvs were purified using metal affinity chromatography (TALON™, BD Biosciences) and dialyzed against 20 mM HEPES, pH 7.0, 0.5 M NaCl, 5% glycerol. Purity was evaluated following ELISA, protein staining and western blot analysis.

Protein gel analysis: Samples were mixed with 2× sample buffer (62.5 mM Tris pH 6.8, 10% (v/v) glycerol, 2% (w/v) SDS, 5% (v/v) β-mercapthethanol, 1% (w/v) bromophenol blue), boiled for 5 minutes and subjected to denaturing polyacrylamide gel electrophoresis (10%) in the presence of SDS (Laemmli, U. K., 1970). Proteins were subsequently visualized following Coomassie Blue staining (SimplyBlue™ SafeStain, Invitrogen).

Western blot analysis—Gels were run as described above, transferred to PVDF membrane (PolyScreenR, Perkin Elmer) and proteins detected following incubation with specific primary antibodies, followed by incubation with the complementary HRP-secondary antibody. Immunocomplexes were detected following chemiluminescence (Western Lightning Plus, Perkin Elmer) and film autoradiography (Hyperfilm, Amersham).

ELISA—Various conformers of SYN or buffer (50 μl; 10 μg/ml) were plated onto microtiter wells and incubated 16 hours at 4° C. Wells were rinsed with ultra-pure water (H2O) to remove unbound antigen, blocked (Roche Blocking Reagent) and incubated with scFv for 2 hours at room temperature. Unbound scFv was removed following 5× washes in buffer containing 50 mM Tris, pH 8.0, 150 mM NaCl, 0.1% Tween 20 (TBST) and 1×H₂O rinse. Antibody:antigen complexes were detected following incubation with HRP-conjugated antibodies ( 1/1000; M2-Flag-HRP or HA-HRP) for 2 hours at room temperature, TBST/H₂O washes and TMB/Peroxidase chromogenic detection at 450 nm (TMB Microwell Peroxidase Substrate System, KPL).

Linear Peptide Mapping—Biotin-conjugated 15 amino acid peptides spanning the entire SYN sequence were synthesized (American Peptide Company; Sunnyvale, Calif.). Peptides were plated onto streptavidin-coated microtiter wells (Express Capture Streptavidin Coated Plates, Express Biotech International; Thurmont, Md.) and exposed to various scFvs. Antigen:antibody interactions were detected as described above.

### Example 5

### Results

To develop a method to efficiently deliver antibodies capable of affecting Parkinson's disease, the ability of rAAV2 vectors expressing scFv secretion constructs were evaluated. For this study, scFvs targeted to prions (PrP) were used. These vectors were delivered directly to the CNS of susceptible C57Bl/6 mice that were subsequently inoculated intraperitoneal (IP) with the RML stain of murine-adapted scrapie. Three novel PrP-specific scFvs were used, PrP 3:3, PrP 6:4, and PrP 6:6, isolated from a human combinatorial phage display library (Haidaris, C. G., et al., 2001) and a scFv version of a Fab termed D18. A scFv, designated Phe and specific to an irrelevant antigen, phenobarbital, was included as a negative control (Malone, J. and M. A. Sullivan, 1996). Each scFV contained a murine Ig Kappa secretory signal for efficient eukaryotic secretion and a c-myc epitope to facilitate detection (FIG. 9 and FIG. 10). Analysis of the brain tissue revealed that the vast majority (>95%) of rAAV2 transduced cells expressing scFvs were neuronal. In addition to scFv expression localized to site of vector instillation, expression from rAAV2 delivery at sites distal was observed (FIG. 11 and FIG. 12). For comparison, expression from a unilaterally delivered rAAVGFP, encoding a cell retained marker gene, was detectable bilaterally in neuronal processes >4 mm from injection site. Bilateral expression of unilaterally delivered scFvs was also detected thus signifying an extended therapeutic paracrine.

Materials and Methods

Phage library Panning: The naïve human combinatorial scFv phage display library, designated pAP-III6, was selected against mouse PrP (rec Mo PrP) (Prionics, AG) using previously described methodologies (Malone, J. and M. A. Sullivan, 1996).

Viral vectors construction: The sequences containing only the V_{L}, linker, and V_{H}, were excised from the AP-III6 phagmid vector with Sal I digest, blunted by Large Fragment Polymerase (Invitrogen), and Hind III digest and sub-cloned into pSecTag2B (Invitrogen) linearized by Hind III and Eco RV digest. The pSecTag2B vector target scFv for secreted expression by fusion to the murine Ig κ-chain secretion signal to the N-terminus each scFv antibody. The murine Ig K-chain secretion signal has demonstrated efficacy for efficient targeting of proteins to secretory pathway (Coloma, M. J., et al., Novel vectors for the expression of antibody molecules using variable regions generated 1992). The pSecTag2 will also append a myc and hexahistidine tag efficient detection and purification. For rAAV2 packaging the pSecTag2B scFv expression cassettes will be cloned into the rAAV2 packaging vector, pFBGR, via blunted NotI sites flanked by AAV ITRs.

Recombinant adeno-associated viruses serotype 2 (rAAV2) packaging: Packaging was performed using methodologies developed by and in collaboration with the laboratory of Dr. Robert Kotin, Laboratory of Biochemical Genetics, National Heart, Lung, and Blood Institute, National Institute of Health, Bethesda, Md. (Urabe, M., et al. 2002).

In vitro Transductions: HEK 293 cells were plated to 80% confluency overnight. Transduced with 1 μl AAV scFv (4.5×10⁸ g.f.u.:expressing units E.U.). Conditioned media was harvested 72 hours post-transduction. The HEK 293 cell line was originally obtained from American Type Culture Collection and will be maintained in Dulbecco's modified Eagle medium (DMEM)-F12 supplemented with 10% fetal bovine serum (FBS).

Enzyme Linked Immunosorbant Assay (ELISA): Microtiter plates coated with PrPc (Alicon AG); blocked with SPOTS blocking buffer (Sigma), challenged with conditioned media. Binding of scFv was detected by anti-Myc HRP conjugate antibody (Invitrogen). HRP activity was detected using 3,3′,5,5′ tetramethylbenzidine (Kirkegaard and Perry Laboratories).

Stereotaxic Surgery: Under surgical plane of Avertin anesthesia, mice were placed in an ASI stereotaxic apparatus and a longitudinal incision of the soft tissues of the skull was performed to expose bregma and lambda sutures. Using a fine Dremel drill, a burr hole approximately 300 μm in diameter is drilled exposing the dura under 40× magnification of an Olympus surgical microscope. A stereotaxic injection is performed using a frame-mounted micromanipulator, holding a microprocessor controlled pump with a Hamilton syringe and a 33 GA needle. 5 μl rAAV2 scFv (2.25×10⁹ g.f.u.:expressing units E.U.) delivered per bilateral injection to striatum stereotaxic coordinates relative to Bregma (+0.4 mm, +/−2.5 mm, −2.5 mm) and thalamus (−2.0 mm, +/−1.5 mm, −3.0 mm) at 0.2 μl/min totaling (9×10⁹ g.f.u.:expressing units E.U.) per mouse.

Immunohistochemistry: Under deep Avertin anesthesia, mice were transcardially perfused with 4% PFA at times indicated post-AAV delivery. Harvested tissue was equilibrated in 30% sucrose and sectioned on sliding microtome to 35 μm sections. Tissue will be blocked with 10% normal goat serum in PBS with 0.1% triton X-100 and probed with primary antibody Myc-tag polyclonal antibody (Cell Signaling) and secondary antibody Peroxidase-conjugated AffiniPure Goat Anti-Rabbit IgG (H+ L) (Jackson ImmunoResearch Laboratories) and developed with 3,3′-diaminobenzidine (DAB) substrate kit for peroxidase (Vector Laboratories) for DAB staining or probed with primary antibodies Myc-tag polyclonal antibody (Cell Signaling), mouse anti-neuronal nuclei (NeuN) monoclonal antibody (Chemicon International), secondary antibodies Alexa Fluor 594 goat anti-rabbit IgG (H+ L) and Alexa Fluor 488 goat anti-mouse IgG (H+ L) (Invitrogen) and 4′,6-Diamidino-2-phenylindole dihydrochloride (DAPI) (Sigma) for fluorescent images.

Microscopy: Performed using our Olympus Provis equipped with a SpotRT camera and MCID software for DAB images and Zeiss Axioscop equipped with a Photometrics CoolSNAP FX camera for fluorescent images.

Immunoprecipitation: Mice were euthanized by cervical dislocation under Avertin anesthesia. Striatum and thalamus were micro-dissected and homogenized in lysis buffer (10 mM trizma base (pH 8.0), 1% triton X-100, 5 mM EDTA, 1 mM iodoacetamide, 150 mM NaCl, 1 mM PMSF and 1× protease inhibitor cocktail (Sigma)). A total of 200 mg of homogenate was diluted 1:20 in lysis buffer and incubated rotating for 16 hours at 4° C. with 4 mg of Myc-Tag 9B11 monoclonal antibody (Cell Signaling). Protein A beads (80 ml of slurry) were added and incubated rotating 1 hour at 4° C. Bead complexes were washed 6 times with lysis buffer and resuspended in 50 μl Laemmli sample buffer and incubation at 100° C. for 5 min. A total of 20 ml of buffer was loaded per lane were electrophoresed on 12% polyacrylamide gel, transferred to PVDF membrane and probed with Myc-Tag polyclonal antibody (Cell Signaling) followed by Peroxidase-conjugated AffiniPure Goat Anti-Rabbit IgG (H+ L) (Jackson ImmunoResearch Laboratories) and chemiluminescent IRP detection (Pierce Super Signal West Dura) followed by exposure to BioMax film (Kodak) and developed (XO-Mat).

PrPsc Challenge: One month post rAAV2 delivery. 50 μl 1% PrPsc infected brain homogenate (approximately 10⁶ ID₅₀ of Rocky Mountain Laboratories (RML) murine adapted prions in PBS delivered intraperitoneally.

Behavior: Rotarod performance was assessed using an Accurotor accelerating rotarod (AccuScan Instruments) with the rod accelerating from 0-30 rpm in 4 minutes. At each time-point mice performed 3 training trials (each separated by 5 minutes) followed by 3 evaluation trials (each separated by 5 minutes) each consisting of the rod starting at 0 rpm and accelerating to 30 rpm over a period of 4 minutes. The time in seconds from initiation of rotation until fall were collected for all mice during each of the three evaluation trials. Performance initiated 24 hours prior to PrPsc challenge and repeated monthly for five months then evaluated weekly.

Clinical Examinations: Clinical criteria were adapted from previously described methodology (Dickinson, A. G., et al. 1968). Exams were performed weekly beginning at week 20 post-PrPsc inoculation. Each mouse was observed for 2 minutes and evaluated for the following clinical symptoms of murine prion disease onset; ataxia, awkward body posture, suppressed righting reflex, extreme in-coordination, flaccid paralysis of hind-limbs, urinary incontinence, malleable plastic tail, swollen inflamed penis, and nestlet under utilization. Each category was scored 0-3 for each mouse. Incubation period was determined as time from PrPsc inoculation until mice reached moribund status.

Proteinase K Immunohistochemistry (Histo Blot): Mice were euthanized by cervical dislocation under Avertin anesthesia. Unfixed cryostat brain sections (12 μm) on glass will be blotted onto nitrocellulose membranes (Taraboulos, A., et al., 1992). Histoblots will then be treated with Proteinase K (200 μg/ml) for 1 hour at 37° C., stopped with Phenylmethylsulphonylfluoride (PMSF) (100 mM) for 30 minutes at room temperature, and denatured with 3M guanidine isothiocyanate (GDN) prior to development with recombinant anti-PrP Fab HuM-D18 (InPro Biotechnology) followed by anti-human Ig G (H+ L) AP conjugate (Promega) and developed with 5-Bromo-4-chloro-3-indoxyl-phosphate, nitroblue tetrazolium (BCIP/NBT) phosphatase substrate (Kirkegaard and Perry Laboratories). Densitometry performed with UVP imaging system.

Proteinase K Immunoblot analysis: Mice will be euthanized by cervical dislocation under Avertin anesthesia. Brain homogenates 10% were prepared in PBS containing 1% Triton X-100 and 1% sodium deoxycholate (DOC). Proteinase K (1 μg/50 μg total protein) was added to the lysates, and the samples will be incubated at 37° C. for 30 min. Reactions terminated by the addition of Laemmli sample buffer and incubation at 100° C. for 10 min. A total of 10 μg of brain homogenate per lane were electrophoresed on 12% polyacrylamide gel, transferred to PVDF membrane and probed with anti-PrPc antibody (M-20, Santa Cruz Biotechnology) followed by Peroxidase-conjugated AffiniPure Goat Anti-Rabbit IgG (H+ L) (Jackson ImmunoResearch Laboratories). Chemiluminescent HRP detection (Pierce Super Signal West Dura) and images were obtained with UVP imaging system.

Surface Plasmon Resonance: Performed on BIACORE 2000. A total of 200 RU of PrPc was covalently linked to CM5 chip via amine coupling. Analyte (scFv) concentrations range from 0 to 7×10⁻⁷ g. Data fit to 1:1 Langmuir binding curve. ScFv purified by Immobilized metal ion affinity chromatography (IMAC) Talon resin (clontec) prior to FPLC size exclusion chromatography (SEC) using Bioselect SEC 125-5 column (Biorad).

Cardiac puncture blood draw: Under surgical plane of Avertin anesthesia, 200 μl of whole-blood was withdrawn from left ventricle.

Serum preparation: Collected whole-blood will be allowed to coagulate at room temperature for 4 hours. Coagulated blood will be pelleted at 4000 rpm in Heraeus biofuge. Clarified serum is aspirated from pellet and stored at 4° C. until analysis.

Anti-scFv Endpoint Titers: Microtiter plates coated with purified scFv (50 μg/ml); blocked with SPOTS blocking buffer (Sigma), challenged with serial 3-fold dilutions of sera. Binding of murine Ig G was detected by Peroxidase-conjugated AffiniPure Goat Anti-Mouse IgG (H+ L) (Jackson ImmunoResearch Laboratories)). HRP activity was detected using 3,3′,5,5′ tetramethylbenzidine (Kirkegaard and Perry Laboratories). Endpoint titers were determined using a scatter plot with o.d. values on the y-axis and dilution⁻¹ on the x-axis, where the x-axis scale was logarithmic. A logarithmic curve fit was applied to each individual dilution series and the point where the curve fit intersects the positive/negative cutoff value was determined.

Sequences

SEQ ID NO:1

MDVFMKGLSKAKEGVVAAAEKTKQGVAEAAGKTKEGVLYVGSKTKEGVVH
GVATVAEKTKEQVTNVGGAVVTGVTAVAQKTVEGAGSIAAATGFVKKDQL
GKNEEGAPQEGILEDMPVDPDNEAYEMPSEEGYQDYEPEA

SEQ ID NO:2

GAPQEGILEDMPVDP

SEQ ID NO:3

MPVDPDNEAYEMPSE

SEQ ID NO:4

VTGVTAVAQKTVEGA

SEQ ID NO:5

DYKDDDDK

SEQ ID NO:6 scFv3

gcgatgccagcattcctgacgacgatacggagctgctgcgcgattacgta
aagaagttattgaagcatcctcgtcagtaaaaagttaatcttttcaacag
ctgtcataaagttgtcacggccgagacttatagtcgctttgtttttattt
tttaatgtatttgtacatggagaaaatcatatgaaaaagacagctatcgc
gattgcagtggcactggctggtttcgctaccgttgcgcaagctgactaca
aggacgacgatgacaagctttcctatgagctgacacagccaccctcagcg
tctgggacccccgggcagagggtcaccatctcttgttctggaagcagctc
caacatcggaagtaattatgtatactggtaccagcagctcccaggaacgg
cccccagactcctcatctataggaataatcagcggccctcaggggtccct
gaccgattctctggctccaagtctggcacctcagcctccctggccatcag
tgggctccggtccgaggatgaggctgattattactgtgcagcatgggatg
acagtctgaatggtccggtattcggcggagggaccaagctcaccgtccta
ggtgagggtaaatcttccggatctggttccgaatccaaagctagcgaggt
gcagctggtgcagtctgggggaggcttggtaaagcctggggggtccctta
gactctcctgtgcagcctctggattcactttcagtaacgcctggatgagc
tgggtccgccaggctccagggaaggggctggagtgggttggccgtattaa
aagcaaaactgatggtgggacaacagactacgctgcacccgtgaaaggca
gattcaccatctcaagagatgattcaaaaaacacgctgtatctgcaaatg
aacagcctgaaaaccgaggacacagccgtgtattactgtacctccccaga
gacactctactggggccagggcaccctggtcaccgtctcctcagtcgacc
cattcgtttctgaatatcaaggccaatcgtctgacctgcctcaacctcct
gtcaatgctggcggcggctctgagggaggcggttccggtggtggctctgt
cc

SEQ ID NO:7 scfv4

gatgcagcattcctgacgacgatacggagctgctgcgcgattacgtaaag
aagttattgaagcatcctcgtcagtaaaaagttaatcttttcaacagctg
tcataaagttgtcacggccgagacttatagtcgctttgtttttatttttt
aatgtatttgtacatggagaaaatcatatgaaaaagacagctatcgcgat
tgcagtggcactggctggtttcgctaccgttgcgcaagctgactacaagg
acgacgatgacaagctttcttctgagctgactcaggaccctgctgtgtct
gtggccttgggacagacagtcaggatcacatgccaaggagacagcctcag
aagctattatgcaagctggtaccagcagaagccaggacaggcccctatac
ttgtcatctatggtaacaataatcggccctcagggatcccagaccgattc
tctggctccagtctaggaaacactgcttccttgaccatcactggggctca
ggcggacgatgaggctgactattactgtaactcccgggacagtagggcta
gacatgttttattcggcggagggaccaaggtgaccgtcctaggtgagggt
aaatcttccggatctggttccgaatccaaagctagcgaggtgtagctggt
gcagtctgggggaggcgtggtcaagcctggagggtccctgagactctcct
gtgaagcctctggattcatactcagtgactattacatgacctggatccgc
caggcaccagggaaggggctggagtggcttgcagtcattgatattactag
tagttacacaaactacgcagactctgtgaagggccgcttcaccatctcca
gagacaacgccaagaactcagtgtatctgcaaatgaacagcctgagagcc
gaggacacggccgtgtattactgtgcgaggctggaatctggcttctttga
ctactggggccagggcaccctggtcaccgtctcctcagtcgacccattcg
tttctgaatatcaaggccaatcgtctgacctgcctcaacctcctgtcaat
gctggcggcggctctgagggaggcggttccggtggtggctctgtccgggg
a

SEQ ID NO:8 scFv5

cgatgcagcattcctgacgacgatacggagctgctgcgcgattacgtaaa
gaagttattgaagcatcctcgtcagtaaaaagttaatcttttcaacagct
gtcataaagttgtcacggccgagacttatagtcgctttgtttttattttt
taatgtatttgtacatggagaaaatcatatgaaaaagacagctatcgcga
ttgcagtggcactggctggtttcgctaccgttgcgcaagctgactacaag
gacgacgatgacaagcttcagtctgccctgactcagcctgcctccgtgtc
tgggtctcctggacagtcgatcaccatctcctgcactggaacctacagtg
acattgctagttataaccacgtctcctggtaccaacaacacccaggcaaa
gcccccaaactcataatttttgaagtcgcttatcggccctcaggggtctc
ttatcgcttctctggctccaagtctggcaacacggcctccctgaccatct
ctgggctccaggctgaggacgaggctgattattactgcagttcatataca
accagccgcacttatgtcttcggagctgggaccaagctcaccgtcctagg
tgagggtaaatcttccggatctggttccgaatccaaagctagcgaggtgc
agctggtgcagtctggaggaggcttgatccagcctggggggtccctgaaa
ctctcctgtgcagcctctgggttcaccttcagtggctctgctatgcactg
ggtccgccaggcttccgggaaagggctggagtgggttggccgtattagaa
gcaaagctaacagttacgcgacagcatatgctgcgtcggtgaaaggcagg
ttcaccatctccagagatgattcaaagaacacggcgtatctgcaaatgaa
cagcctgaaaaccgaggacacggccgtgtattactgtactagacatcgag
gagggcccattgactggggccagggcaccctggtcaccgtctcctcagtc
gacccattcgtttctgaatatcaaggccaatcgtctgacctgcctcaacc
tcctgtcaatgctggcggcggctctgagggaggcggttccggtggtggct
ctgtccggga

SEQ ID NO:9 scFv6

cgatgcagcattcctgacgacgatacggagctgctgcgcgattacgtaaa
gaagttattgaagcatcctcgtcagtaaaaagttaatcttttcaacagct
gtcataaagttgtcacggccgagacttatagtcgctttgtttttattttt
taatgtatttgtacatggagaaaatcatatgaaaaagacagctatcgcga
ttgcagtggcactggctggtttcgctaccgttgcgcaagctgactacaag
gacgacgatgacaagcttcagtctgtgctgactcagccgccctcagtgtc
tggggccccaggtcagagggtcgccatctcctgcactgggaccagctccg
acatcgggacaggttatgatgtaaattggtaccagcatcttccaggaaca
gcccccagactcctcatctatggtaatacctatcgaccctcaggggtccc
tgaccgattctctgcctccactggctccaagtctggcacctcagcctccc
tggtcatcactgacctccaggctgaagatgagggtgattattactgccag
tcttttgacaacagcctgagaggttcggtgttcggcggagggaccaaggt
gaccgtcctaggtgagggtaaatcttccggatctggttccgaatccaaag
ctagcgaggtgcagctggtgcagtctgggggaggcgtggtcaagcctgga
gggtccctgagactctcctgtgaagcctctggattcatactcagtgacta
ttacatgacctggatccgccaggcaccagggaaggggctggagtggcttg
cagtcattgatattactagtagttacacaaactacgcagactctgtgaag
ggccgcttcaccatctccagagacaacgccaagaactcagtgtatctgca
aatgaacagcctgagagccgaggacacggccgtgtattactgtgcgaggc
tggaatctggcttctttgactactggggccagggcaccctggtcaccgtc
tcctcagtcgacccattcgtttctgaatatcaaggccaatcgtctgacct
gcctcaacctcctgtcaatgctggcggcggctctgagggaggcggttccg
gtggtggctctggttcc

SEQ ID NO:10 scfv7

gatgcagcattcctgacgacgatacggagctgctgcgcgattacgtaaag
aagttattgaagcatcctcgtcagtaaaaagttaatcttttcaacagctg
tcataaagttgtcacggccgagacttatagtcgatttgtttttatttttt
aatgtatttgtacatggagaaaatcatatgaaaaagacagctatcgcgat
tgcagtggcactggctggtttcgctaccgttgcgcaagctgactacaagg
acgacgatgacaagcttcagtctgccctgactcagcctcgctcagtgtcc
gggactcctggacagtcagtcaccatctcctgcactggaaccaacagtga
tgttggtcgttatgactatgtctcgtggtaccaacagtattcagacaaag
cccccaaactcatcatttatgatgactttaagcggccctcaggggtccct
gatcgcttctctggctccaagtctggcaacacggcctccctgaccatctc
tgggctccaggctgaggatgaggctgattattactgcagctcatatacaa
gcagcagccgggcgttcggcggagggaccaaggtgaccgtcctaggtgag
ggtaaatcttccggatctggttccgaatccaaagctagcgaggtgcagct
ggtgcagtctgaagctgaggtgaggaagcctggggcctcagtgaaggtct
cctgcaaggcttctggttttcactttaccagctacggtttcatgtgggtg
cggcaggcccctggacaagggcttgagtggatgggttggatcagcgctta
caatggtaacacaaagtcggctcagaagttccagggcagactcaccatga
ccactgacacatccacgaacacagcccacatggagttgatgagcctgaga
tctgacgacacggccgtgtattactgtgcgagagattctgcgactctaat
agcctctcgacaggggggctctgactactggggccagggcaccctggtca
ccgtctcctcagtcgacccattcgtttctgaatatcaaggccaatcgtct
gacctgcctcaacctcctgtcaatgctggcggcggctctgagggaggcgg
ttccggtggtggctctggtccgggga

SEQ ID NO:11 scfv8

cgatgcagcattcctgacgacgatacggagctgctgcgcgattacgtaaa
gaagttattgaagcatcctcgtcagtaaaaagttaatcttttcaacagct
gtcataaagttgtcacggccgagacttatagtcgctttgtttttattttt
taatgtatttgtacatggagaaaatcatatgaaaaagacagctatcgcga
ttgcagtggcactggctggtttcgctaccgttgcgcaagctgactacaag
gacgacgatgacaagcttgatgttgtgatgactcagtctccaggcaccct
gtctttgtctccaggggaaagagccaccctctcctgcagggccagtcaga
gtgttagcatcgtctatttagcctggtaccagcagaaacctggccaggct
cccaggctcctcatctatggtgcatccaccagggccactggtatcccagc
caggttcagtggcagtgggtctgggacagagttcactctcaccatcagca
gcctgcagtctgaagattttgcggtttattactgtcagcagtataatgac
tggttcactttcggcggagggaccaaagtggatatcaaagagggtaaatc
ttccggatctggttccgaatccaaagctagcgaggtgcagctggtgcagt
ctgggggaggcgtggtcaagcctggagggtccctgagactctcctgtgaa
gcctctggattcatactcagtgactattacatgacctggatccgccaggc
accagggaaggggctggagtggcttgcagtcattgatattactagtagtt
acacaaactacgcagactctgtgaagggccgcttcaccatctccagagac
aacgccaagaactcagtgtatctgcaaatgaacagcctgagagccgagga
cacggccgtgtattactgtgcgaggctggaatctggcttctttgactact
ggggccagggaaccctggtcaccgtctcctcagtcgacccattcgtttct
gaatatcaaggccaatcgtctgacctgcctcaacctcctgtcaatgctgg
cggcggctctgagggaggcggttccggtggtggctctgtcc

SEQ ID NO:12 scFv10

gatgcagcattcctgacgacgatacggagctgctgcgcgattacgtaaag
aagttattgaagcatcctcgtcagtaaaaagttaatcttttcaacagctg
tcataaagttgtcacggccgagacttatagtcgctttgtttttatttttt
aatgtatttgtacatggagaaaatcatatgaaaaagacagctatcgcgat
tgcagtggcactggctggtttcgctaccgttgcgcaagctgactacaagg
acgacgatgacaagcttcagtctgtgctgactcagccgccctcagcgtct
gggacccccgggcagagggtcaccatctcttgttctggaagcagctccaa
catcggaagtaatactgtaaactggtaccagcagctcccaggaacggccc
ccaaactcctcatctatagtaataatcagcggccctcaggggtccctgac
cgattctctggctccaagtctggcacctcagcctccctggccatcagtgg
gctccagtctgaggatgaggctgattattactgtgcagcatgggatgaca
gcctgagtggtgtggtattcggcggagggaccaagctgaccgtcctaggt
gagggtaaatcttccggatctggttccgagtccaaagctagccaggtgca
gctgcagtctggagctgaggtgaagaagcctggggcctcagtgaaagtct
cctgcaaggcttctggttacacgtttaacactcatggtttcagctgggta
cgacaggcccctggacaagggcttgagtggatgggatggatcagcgcatc
caatggtaacacaaagtatccccagaacctccagggcagagtcaccatga
ctattgacacattcacgaccacagcatacctggaactgaggagcctgcga
tctgacgacacggccgtgtattattgtgtgagagatagaacagattacta
tgttccggggacttttgatcccttatatgggccctttgattactggggcc
agggcaccctggtcaccgtctcctcagtcgacccattcgtttctgaatat
caaggccaatcgtctgacctgcctcaacctcctgtcaatgctggcggcgg
ctctgagggaggcggttccggtggtggctctg

SEQ ID NO:13 scFv14

cgatgcagcattcctgacgacgatacggagctgctgcgcgattacgtaaa
gaagttattgaagcatcctcgtcagtaaaaagttaatcttttcaacagct
gtcataaagttgtcacggccgagacttatagtcgctttgtttttattttt
taatgtatttgtacatggagaaaatcatatgaaaaagacagctatcgcga
ttgcagtggcactggctggtttcgctaccgttgcgcaagctgactacaag
gacgacgatgacaagcttcagtctgccctgactcagcctgcctccgtgtc
tgggtctcctggacagtcgatcaccatctcctgcactggaaccagcagtg
acgttggtacttataattatgtctcttggtatcaacaatatccaggcaaa
gcccccaaactcctgatttatgatgtcgttaagcggccctcaggggtttc
taatcgcttctctggctccaagtctggcagcacggcctccctgaccatct
ctgggctccaggctgaggacgaggctgattattactgcagctcatataca
accaggaacactcgggtattcggcggagggaccaagctcaccgtcctagg
tgagggtaaatcttccggatctggttccgaatccaaagctagccaggttc
agctggtgcagtctgggggaggcgtggtccagcctgggaggtccctgaga
ctctcctgtgcagcctctggattcaccttcagtagctatggcatgcactg
ggtccgccaggctccaggcaaggggctggagtgggtggcagttatatcat
atgatggaagtaataaatactatgcagactccgtgaagggccgattcacc
atctccagagacaattccaagaacacgctgtatctgcaaatgaacagcct
gagagctgaggacacggctgtgtattactgtgcgaagaacttgggtggta
actccggtggggtggcctactggggccagggcaccctggtcaccgtctcc
tcagtcgacccattcgtttctgaatatcaaggccaatcgtctgacctgcc
tcaacctcctgtcaatgctggcggcggctctgagggaggcggttccggtg
gtggctctgtcc

SEQ ID NO:14 scFv15

cgatgcagcattcctgacgacgatacggagctgctgcgcgattacgtaaa
gaagttattgaagcatcctcgtcagtaaaaagttaatcttttcaacagct
gtcataaagttgtcacggccgagacttatagtcgctttgtttttattttt
taatgtatttgtacatggagaaaatcatatgaaaaagacagctatcgcga
ttgcagtggcactggctggtttcgctaccgttgcgcaagctgactacaag
gacgacgatgacaagcttcaggctgtgctcactcagccgtcttccctctc
tgcatctcctggagcatcagccagtctcacctgcaccttgcgcagtggca
tcaatgttggtacctacaggatatactggtaccagcagaagccagggagt
cctccccagtatctcctgaggtacaaatcagactcagataagcagcaggg
ctctggagtccccagccgcttctctggatccaaagatgcttcggccaatg
cagggattttactcatctctgggctccagtctgaggatgaggctgactat
tactgtatgatttggcacagcagcgcttatgtcttcggaactgggaccaa
gctgaccgtcctaggtgagggtaaatcttccggatctggttccgaatcca
aagctagcgaggtgcagctggtggagtctgggggaggcttggtacagcct
ggggggtccctgagactctcctgtgcagcctctggattcacctttagcag
ctatgccatgagctgggtccgccaggctccagggaaggggctggagtggg
tctcagctattagtggtagtggtggtagcacatactacgcagactccgtg
aagggccggttcaccatctccagagacaattccaagaacacgctgtatct
gcaaatgaacagcctgagagccgaggacacggccgtatattactgtgcga
gcggtagcagctcgtccttgggctactactactacggtatggacgtctgg
ggccaagggaccacggtcaccgtctcctcagtcgacccattcgtttctga
atatcaaggccaatcgtctgacctgcctcaacctcctgtcaatgctggcg
gcggctctgagggaggcggttccggtggtggctctggt

SEQ ID NO:15 scfv16

cgatgcagcattcctgacgacgatacggagctgctgcgcgattacgtaaa
gaagttattgaagcatcctcgtcagtaaaaagttaatcttttcaacagct
gtcataaagttgtcacggccgagacttatagtcgctttgtttttattttt
taatgtatttgtacatggagaaaatcatatgaaaaagacagctatcgcga
ttgcagtggcactggctggtttcgctaccgttgcgcaagctgactacaag
gacgacgatgacaagcttcagtctgccctgactcagcctgcctccgtgtc
tgggtctcctggacagtcgatcaccatctcctgcactgaaaccggcagtg
acgttggtggttatcactatgtctcctggtatcaacaccacccagacaaa
gcccccaaactcataatttatgatgactttaagcggccctcaggggtttc
tgatcgcttctctggctccaagtctggcaacacggcctccctgaccatct
ctgggctccgggctgaggacgaggctgattattactgcggctcatttaca
agcagtgccccttggttgttcggcggagggaccaagctgaccgtcctagg
tgagggtaaatcttccggatctggttccgaatccaaagctagcgaggtgc
agctggtgcagtctgggggaggcttggtacagcctggggggtccctgaga
ctctcctgtgcagcctctggattcacctttagcagttatgccatgagctg
ggtccgccaggctccagggaaggggctggagtgggtctcagcaattagtg
gtagtggtagtatcgcatactacgcagactccgtgaagggccggttcacc
atctccagagacaattccaagaacacgctgtatctgcaaatgaacagcct
gagagtcgaggacacggccgtatattactgtgcacgacgagctactggaa
cctcgtactactttgactactggggccagggcaccctggtcaccgtctcc
tcagtcgacccattcgtttctgaatatcaaggccaatcgtctgacctgcc
tcaacctcctgtcaatgctggcggcggctctgagggaggcggttccggtg
gtggctctgtcc

### REFERENCES

Coloma, M. J., et al., Novel vectors for the expression of antibody molecules using variable regions generated by polymerase chain reaction. J Immunol Methods, 1992. 152(1): p. 89-104.
Dickinson, A. G., V. M. H. Meikle, and H. Fraser, Identification of a gene which controls the incubation period of some strains of scrapie agent in mice. J. Comp. Path., 1968. 78: p. 293-299.
Haidaris, C. G., et al., Recombinant human antibody single chain variable fragments reactive with Candida albicans surface antigens. J Immunol Methods, 2001. 257(1-2): p. 185-202.
Malone, J. and M. A. Sullivan, Analysis of antibody selection by phage display utilizing anti-phenobarbital antibodies. J Mol Recognit, 1996. 9(5-6): p. 738-45.
Malone, J. and M. A. Sullivan, Analysis of antibody selection by phage display utilizing anti-phenobarbital antibodies. J Mol Recognit, 1996. 9(5-6): p. 738-45.
Taraboulos, A., et al., Regional mapping of prion proteins in brains. Proc Natl Acad Sci U S A, 1992: p. 7620-7624.
Urabe, M., C. Ding, and R. M. Kotin, Insect cells as a factory to produce adeno-associated virus type 2 vectors. Hum Gene Ther, 2002. 13(16): p. 1935-43.

What is claimed is:

1. An alpha-synuclein antibody, wherein the antibody specifically binds alpha-synuclein in the native monomer form.

2. The antibody of claim 1, wherein the antibody is a single chain antibody (scFv).

3. The antibody of claim 1, wherein the antibody is a humanized antibody.

4. The antibody of claim 1, wherein the antibody is a monoclonal antibody.

5. A kit comprising the antibody of claims 1-4.

6. An alpha-synuclein antibody, wherein the antibody specifically binds alpha-synuclein bonded to dopamine quinone.

7. The antibody of claim 6, wherein the antibody is a single chain antibody (scFv).

8. The antibody of claim 6, wherein the antibody is a humanized antibody.

9. The antibody of claim 6, wherein the antibody is a monoclonal antibody.

10. A kit comprising the antibody of claims 6-9.

11. An alpha-synuclein antibody, wherein the antibody specifically binds alpha-synuclein in the oligomeric or aggregated form.

12. The antibody of claim 11, wherein the antibody is a single chain antibody (scFv).

13. The antibody of claim 11, wherein the antibody is a humanized antibody.

14. The antibody of claim 11, wherein the antibody is a monoclonal antibody.

15. A kit comprising the antibody of claims 11-14.

16. A diagnostic assay for Parkinson's disease in a subject, comprising contacting a sample from the subject with one or more antibodies or fragments thereof specific for one or more alpha-synuclein conformers.

17. The assay of claim 16, wherein the sample is a blood, CSF, or urine sample.

18. The assay of claim 16, wherein the antibody specifically binds a native monomeric alpha-synuclein.

19. The assay of claim 18, wherein the antibody is a single chain antibody (scFv).

20. The assay of claim 18, wherein the antibody is a humanized antibody.

21. The assay of claim 18, wherein the antibody is a monoclonal antibody.

22. The assay of claim 16, wherein the antibody specifically binds an alpha-synuclein bonded to a dopamine quinone.

23. The assay of claim 22, wherein the antibody is a single chain antibody (scFv).

24. The assay of claim 22, wherein the antibody is a humanized antibody.

25. The assay of claim 22, wherein the antibody is a monoclonal antibody.

26. The assay of claim 16, wherein the antibody specifically binds an oligomeric or aggregated alpha-synuclein.

27. The assay of claim 26, wherein the antibody is a single chain antibody (scFv).

28. The assay of claim 26, wherein the antibody is a humanized antibody.

29. The assay of claim 26, wherein the antibody is a monoclonal antibody.

30. A method of diagnosing Parkinson's disease in a subject, the method comprising:
a. assessing a level of one or more alpha-synuclein conformers in a sample from the subject to be diagnosed; and
b. comparing the level of the alpha-synuclein conformers to a reference standard that indicates the level of the alpha-synuclein conformers in one or more control subjects,
wherein a difference or similarity between the level of the alpha-synuclein conformers and the reference standard indicates that the subject has Parkinson's disease.

31. The method of claim 30, wherein the subject to be diagnosed is a human.

32. The method of claim 30, wherein the subject to be diagnosed is asymptomatic or preclinical for Parkinson's disease.

33. The method of claim 30, wherein the control subject has Parkinson's disease and wherein a similarity between the level of alpha-synuclein conformer and the reference standard indicates that the subject to be diagnosed has Parkinson's disease.

34. The method of claim 30, wherein the control subject does not have Parkinson's disease and wherein a difference between the level of alpha-synuclein conformer and the reference standard indicates that the subject to be diagnosed has Parkinson's disease.

35. The method of claim 30, wherein assessing the level of alpha-synuclein conformer comprises analyzing the alpha-synuclein conformer by one or more techniques chosen from Western blot, immunoprecipitation, enzyme-linked immunosorbent assay (ELISA), radioimmunoassay (RIA), fluorescent activated cell sorting (FACS), two-dimensional gel electrophoresis, mass spectroscopy (MS), matrix-assisted laser desorption/ionization-time of flight-MS (MALDI-TOF), surface-enhanced laser desorption ionization-time of flight (SELDI-TOF), high performance liquid chromatography (HPLC), fast protein liquid chromatography (FPLC), multidimensional liquid chromatography (LC) followed by tandem mass spectrometry (MS/MS), protein chip expression analysis, gene chip expression analysis, and laser densitometry.

36. The method of claim 30, wherein the subject to be diagnosed and the control subject are age-matched.

37. The method of claim 30, wherein the alpha-synuclein conformer is in the native monomer form.

38. The method of claim 30, wherein the alpha-synuclein conformer is bonded to dopamine-quinone.

39. The method of claim 30, wherein the alpha-synuclein conformer is in the oligomeric or aggregate form.

40. A method of monitoring Parkinson's disease progression in a subject, the method comprising comparing a level of alpha-synuclein conformer in a sample obtained from the subject at multiple time points.

41. The method of claim 40, wherein the subject is a human.

42. The method of claim 40, wherein the subject is asymptomatic or preclinical for Parkinson's disease at one or more of the multiple time points.

43. The method of claim 40, wherein the subject has not received medical treatment for Parkinson's disease at or before one or more of the multiple time points.

44. The method of claim 40, wherein the subject has received medical treatment for Parkinson's disease at or before one or more of the multiple time points.

45. The method of claim 40, wherein the subject has been treated with levodopa at or before one or more of the multiple time points.

46. The method of claim 40, wherein the subject has been treated with a neuroprotective agent at or before one or more of the multiple time points.

47. The method of claim 40, wherein the alpha-synuclein conformer is in the native monomer form.

48. The method of claim 40, wherein the alpha-synuclein conformer is bonded to dopamine-quinone.

49. The method of claim 40, wherein the alpha-synuclein conformer is in the oligomeric or aggregate form.

50. A method of monitoring a response to a Parkinson's disease treatment in a subject, the method comprising comparing a level of alpha-synuclein conformer in a sample obtained from the subject at multiple time points during treatment of the subject.

51. The method of claim 50, wherein the subject is a human.

52. The method of claim 50, wherein the subject is asymptomatic or preclinical for Parkinson's disease at one or more of the multiple time points.

53. The method of claim 50, wherein the subject is treated with a neuroprotective agent at or before one of the multiple time points.

54. The method of claim 50, wherein the neuroprotective agent is an acetylcholinesterase inhibitor, a glutamatergic receptor antagonist, an anti-inflammatory, a kinase inhibitor, or divalproex sodium.

55. The method of claim 50, wherein the alpha-synuclein conformer is in the native monomer form.

56. The method of claim 50, wherein the alpha-synuclein conformer is bonded to dopamine-quinone.

57. The method of claim 50, wherein the alpha-synuclein conformer is in the oligomeric or aggregate form.

58. A method of treating Parkinson's disease in a subject, comprising administering to the subject a nucleic acid encoding a single chained antibody (scFv) that specifically binds alpha-synuclein.

59. The method of claim 58, wherein the scFv specifically binds alpha-synuclein monomer.

60. The method of claim 58, wherein the scFv specifically binds alpha-synuclein bonded to dopamine-quinone.

61. The method of claim 58, wherein the scFv specifically binds alpha-synuclein in the oligomeric or aggregate form.
